(19) United States
(12) Patent Application Publication
(10) Pub. No.: US 2007/0141134 A1
(43) Pub. Date: Jun. 21, 2007

(54) Shielded micelles for polynucleotide delivery

(76) Inventor: Matthew K. Kosak, Salt Lake City, UT (US)

Correspondence Address: MATTHEW K. KOSAK, 272E. New Century Ln #F, Salt Lake City, UT, 84115, US

(21) Appl. No.: 11/305,688

(22) Filed: Dec. 16, 2005

(51) Int. Cl. A61K 48/00; A61K 39/00; A61K 9/127; C12N 15/88; A61K 31/4706

(52) U.S. Cl. 424/450; 424/133.1; 514/44; 435/458; 514/313

(57) ABSTRACT

The present invention relates to compositions that enhance the intracellular delivery of polynucleotides. The present invention is applicable to the fields of gene therapy and oligonucleotide or DNA therapy. Synthetic methods are disclosed, wherein a polynucleotide can be incorporated into the PEG shielded micelle particle to facilitate the delivery of the polynucleotide across a cellular membrane. Incorporation of the polynucleotide into the shielded micelle particle is provided by covalent and non-covalent means. Other cell targeting agents are provided that may also be covalently coupled to the shielded micelle particle to enhance localization in the body. The compositions herein are suitable for pharmaceutical use but are also suitable as transfection agents for in-vitro or in-vivo research. The PEG shielded polynucleotide micelles can provide favorable pharmacokinetic properties such as enhanced uptake into cancer cells, stability against nucleases, high solubility, and non-binding to serum proteins. The invention comprises a novel gene carrier which is shown to be substantially non-toxic and is suitable for parenteral, oral, pulmonary, and transmucosal delivery of polynucleotides.

### RELATED PATENT APPLICATION

This application is a continuation in part of the PCT filed Sep. 15, 2005, entitled “Chloroquine Coupled Compositions and Methods For Their Synthesis”, by inventors Ken M. Kosak and Matthew K. Kosak. The entire contents of the application are incorporated herein.

### TECHNICAL FIELD OF THE INVENTION

This invention relates to the intracellular delivery of polynucleotides. It describes novel compositions that substantially promote the delivery of polynucleotides across the cellular membrane. The compositions described herein are novel pharmaceuticals and are intended to be applied to improving human health through polynucleotide therapy.

### DESCRIPTION OF THE PRIOR ART

The problems facing polynucleotide delivery can roughly be divided into two parts. First, the therapeutic polynucleotide must be formulated in such a way that it can be delivered to the cytoplasm and second, the polynucleotide must reach the cell nucleus intact and fully functional. Polynucleotides do not readily permeate the cellular membrane due to the charge repulsion between the negatively charged membrane and the high negative charge on the polynucleotide. As a result, polynucleotides have poor bioavailability and uptake into cells, typically <1% [Dheur et al, Nucleic Acid Drug Dev., 9:522 (1999), Park et al, J Controlled Release, 93:188 (2003)]. The relatively large size of polynucleotides also creates barriers to delivery. Since most polynucleotides are generally above 5000 they cannot readily diffuse through cellular membranes and uptake into cells is limited primarily to pinocytotic or endocytotic processes. Once inside the cell, polynucleotides can accumulate in lysosomal compartments, limiting their access to the cytoplasm or the nucleus. Parenterally administered polynucleotides are also highly susceptible to rapid nuclease degradation both inside and outside the cytoplasm. Studies show rapid degradation of polynucleotides in blood after i.v. administration, with a half life of about 30 minutes [Geary et al, J. Pharmacol. Exp. Ther. 296:890-897 (2001)]. Current strategies to improve the structural stability and lifetime of polynucleotides in vivo are aimed at the modification of the phosphodiester backbone structure of the polynucleotides to reduce enzymatic susceptibility. Other strategies simultaneously address stability and delivery by the use of DNA condensing agents such as cationic vectors. Cationic vectors have increased uptake of polynucleotides by 20 fold, but toxicity is a problem. The current invention is intended to enhance stability, uptake, and delivery of polynucleotides without the side effects associated with current non-viral vectors.

Current technology for non-viral transfection is based primarily on the condensation of polynucleotides into particles using a cationic delivery system. Cationic delivery can be facilitated by a cationic lipid, a polyamine, a positively charged peptide, a polylysine, or a combination thereof that produces an ionic interaction and subsequent complexation between the cationic group and the negatively charged polynucleotide.

Cationic delivery agents have become the standard for non-viral vector delivery because they are believed to facilitate transport of polynucleotides and to improve serum stability in vivo. It is therefore not surprising that many of the recent developments in the field have focused on modification of the cationic system by combining a proven cationic delivery agent with another moiety. For example, polyethylenimine (PEI) is a proven cationic vector that has been demonstrated to promote transfection without further modification. Since its discovery, many attempts have been made to improve on PEI by conjugation with other targeting agents, such as PEG, transferrin, folic acid, avidin, antibodies, viral proteins, and other cell targeting ligands [Ogris et al. Gene Therapy 6:595-605 (1999), Kircheis et al, J. Gene Med. 1:111-120 (1999)]. Other cationic backbones have been modified similarly as PEI, such as polyamidoamine (PAMAM). However, cationic backbone conjugates have not been successful in overcoming toxicity and none are approved for therapeutic use.

The mode of action of polynucleotide delivery systems is complex and not well understood. However, there are at least two fundamental requirements that must be met for successful transfection. First, the carrier must facilitate the endocytotic uptake of polynucleotides across the cellular membrane, and second, the carrier must facilitate release of the polynucleotides from the lysosomal compartments to the cytoplasm. The condensation of the polynucleotides by the cationic groups is believed to facilitate the endocytotic uptake of polynucleotides by reducing the excess negative charge of the polynucleotide and by the condensation of the polynucleotide into positively charged particles. However, once inside the endosomes, polynucleotides must still overcome the endosomal membrane. Polynucleotides have been shown to be taken up into endosomes but are not subsequently released [K. Kunath et al., J. Controlled Release. 89:113-125 (2003)]. As a result, the polynucleotide never reaches the nucleus or the polynucleotide is degraded in the endosome. To disrupt lysosomes, the addition of lysosome disrupting agents have been required such as chloroquine. Polyethylenimines and polyamidoamine dendrimers such as PAMAM, are also believed to facilitate the escape of polynucleotides from the lysosomal compartments by causing osmotic swelling of the endosomal membrane. Thus, the charge ratio of the cationic group to the polynucleotide has been shown to influence the success rate of transfection.

Protecting the polynucleotide from enzymatic degradation is also an important function of the carrier. It is proposed that the condensation of the polynucleotide into polyplex structures interferes with nuclease activity (Szoka U.S. Pat. No. 6,113,946, Dheur et al 1999). This function has been widely reported with the use of PEI. Encapsulation by cationic delivery agents has been proposed as a protecting mechanism for unmodified diester polynucleotides from 3′ exonuclease activity (Betbeder et al, U.S. Pat. No. 6,214,621). PEI for example, has shown improved stability over other cationic vectors such as cationic lipids (Transfectamine™). Since nucleases are present inside and outside the cell, a negative result of an experiment is often interpreted as degradation of the polynucleotide en route to the nucleus. Since serum stability is a critical impediment to polynucleotide delivery in vivo, this is another reason that many of the current vector strategies include a polycation moiety.

Cationic delivery systems readily form sediments, aggregates, and microscopic precipitates. Since these particles are of microscopic size they are too large to be efficiently taken up by cells, and the loading efficiency and bioavailability of these systems is significantly reduced Polyplexes formed from cationic liposome's and cationic lipids have also been described as unstable (Wheeler et al, U.S. Pat. No. 5,976,567) and bovine serum proteins are notorious for interfering with cationic lipid (Lipofectamine™) and PEI systems. Since particle size cannot be readily controlled, the transfection results from these systems also cannot be easily predicted. In the present composition, the polynucleotide and the polymer can be combined at high nitrogen/phosphate (N/P) ratio to produce clear dispersions and sedimentation is avoided.

The toxicity of cationic systems is indicative of the degree of charge on the cationic polymer or monomer employed but also demonstrates that the charges are exposed to the outside. Polyplexes obtained with PEI and polyamidoimine starburst dendrimer (PAMAM) have significant in vivo toxicity due to interaction with negatively charged erythrocytes and activation of the complement system [(Kircheis et al, Adv. Drug Rev. 53:341-358 (2001)]. The degree of toxicity reportedly is higher for more highly charged species. Cationic liposomes for polynucleotide delivery are well known to be toxic in-vitro and in vivo because they employ cationic lipids such as DODAC. At concentrations as low as 50 uM cationic liposomes killed 90% of HEK 293 cells in 24 hours and polynucleotide complex liposomes up to 70% within 48 hours [Hu et al., Nuc. Acids Res. 30:3632-3641 (2002)].

Since the hybridization of a polynucleotide to a target is highly specific, great efforts have been made in the area of carriers, to preserve the specificity of the polynucleotide (Betbeder et al U.S. Pat. No. 6,214,621). Thus, the polynucleotides in the vast majority of the prior art are described as being associated non-covalently with the carrier so that the polynucleotide can disassociate from the carrier once it reaches the cytoplasm. In Szoka, the polynucleotide is condensed with the cationic backbone polymer (starburst dendrimer) PAMAM. In Sagara (U.S. Pat. No. 6,586,524) the polynucleotide is condensed by a cationic polymer backbone of PEI or polylysine at N/P ratio of 2 to 20. In Park (U.S. Pat. No. 6,177,274) the cationic backbone is also polylysine. In Wang et al, (app #20030144222) the PEI or polylysine is the backbone polymer and pendant CD's associate with the polynucleotide. However, these systems rely on cationic backbone polymers and suffer similar drawbacks as other cationic systems.

Currently, there are no examples of polynucleotide delivery systems in which a polynucleotide is conjugated to a multifunctional PEG. Previously, polynucleotide conjugates have been shown to be ineffective by themselves and still require condensation by additional cationic agents [(Jeong et al, Bioconjugate Chem. 14:477 (2003)]. Conjugation alone does not provide a means for the polynucleotide to enter cells or escape from cellular compartments, nor does it protect the polynucleotide from enzymatic degradation.

Conjugates may cause steric hindrance, preventing the polynucleotide from hybridizing with a target or inhibiting biological activity. However, the present invention demonstrates that incorporation of polynucleotides into amphiphilic, multifunctional PEG micelles is a means for delivering active polynucleotides without cationic agents. In some aspects the addition of cationic agents are even shown to inhibit the function of the present invention and highlight the fact that a novel mechanism is involved.

Shielded micelles comprising multifunctional PEG are discovered to have many surprising and significant advantages over particles made with other PEG's. Multi-functional PEG's have greater shielding than other PEG's and show higher rates of transfection, gene expression, and reduced toxicity.

The present invention provides a rationally designed carrier system that enhances bioavailability, stability, and bioactivity of polynucleotides. Finally, the present invention comprises a shielded PEG micelle which is non-toxic and compositions are described that are suitable for pharmaceutical applications.

There are several fundamental criteria that relate specifically to the function of the successful polynucleotide delivery system. 1) The polynucleotide carrier must facilitate the transport of the polynucleotide across the cellular membrane. 2) The polynucleotide carrier must facilitate escape of the polynucleotide from the lysosomal compartment 3) The polynucleotide carrier must protect the polynucleotide from nuclease degradation, and finally, 4) The polynucleotide carrier should be non-cytotoxic by itself (low background).

To make gene therapy a viable reality, there is presently a need for a non-viral vector system that can overcome the current problems of delivery and be invisible to the body, i.e. non-toxic and non-immunogenic. The present invention enhances bioavailability, intracellular transport, stability, and targeted delivery of polynucleotides with a rationally designed synthetic vector.

### SUMMARY OF THE INVENTION

This invention relates to methods and compositions for the intracellular delivery of polynucleotides and addresses the delivery of therapeutic polynucleotides used in DNA, RNA, and gene therapy. More specifically, this invention relates to delivery of polynucleotides using amphiphilic polymers, comprising multifunctional PEG, spacer, and targeting moieties, which are highly efficient transfection agents and substantially less cytotoxic than existing delivery systems. Delivery of polynucleotides is facilitated by covalent or non-covalent association of the amphiphilic polymers of the invention with polynucleotides to form a shielded micelle. The delivery of polynucleotides across the cellular membrane, their lysosomal escape, and subcellular trafficking is thought to be facilitated by the shielding effect of the amphiphilic polymer units in the micelle. Multi-functional PEG is shown to have significant advantages over linear PEG in the formation of shielded particles. For example, the novel particles are shown to form clear dispersions of the multifunctional PEG polymer and the polynucleotide at high N/P ratio, and enhance bioavailability. The unique properties of the invention are useful for pharmaceutical applications relating to polynucleotide delivery.

### BRIEF DESCRIPTION OF THE DRAWINGS

FIG. 1. Shows the sequence specific reduction in proliferation of human MCF-7 tumor cells by antisense phosphorothioated oligonucleotide (anti-sense ODN, G3139 Genta Inc.) conjugated to pendant PEG (pPEG), and also inhibition of anti-sense activity of particles by complexing with PEI at 4:1, 3:1, and 2.5:1 N/P ratio (EX62604A). Cell viability was assayed via mitochondrial dehydrogenase activity against 3-[4,5-dimethylthiazol-2-yl]-2,5-diphenyl tetrazolium bromide (MTT). Mitochondrial dehydrogenases of viable cells cleave the tetrazolium ring, yielding purple, water insoluble, MTT formazan crystals which are quantified at A570.

FIG. 2. Shows luciferase signal after differing amounts of plasmid PGL-3 DNA were transfected into COS-7 cells with either hyperbranched pPEG-PAM (9:1 mass ratio, m.r.), PAMAM-Monomer G=5 (13:1 m.r.), pPEG-Protamine (100:1 m.r.) with 50 mMPEG-HQ, or Superfect™. Luminescence from duplicates was assayed in cells after 48 hour incubation using Promega lysis buffer and luciferase assay reagent (EXP021605, EXP060505).

FIG. 3. Shows inhibition of condensation (shielding) of DNA by various pPEG-amine conjugates, PEG-PEI (1020H, 1123A, 1123B), PEG-Hdz, PEI only, PEGs only, and pPEG-Protamine (pPEG-Protamine11105C) in the PEI condensation assay (EXP 030405). Also shown are the ineffective shielding of cationic polymers (PEI's), linear PEG-PEI conjugates, of PEG's only, and of pPEG-amine isomers (pPEG-Hdz914B).

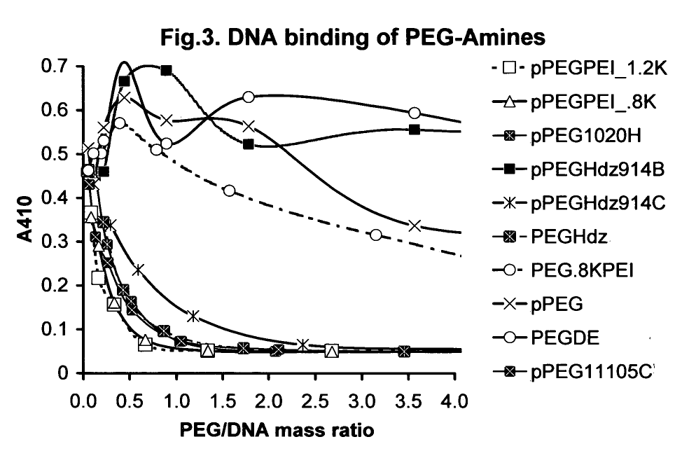

FIG. 4. Shows silencing of luciferase gene in COS-7 cells by interfering dsRNA delivered with pPEG-PEI2K w/wo 50 mM HQ, linear PEG-PEI25K, pPEG-Protamine, pPEG-Protamine/PEG-HQ (50 mM and 150 mM) mixed micelle, commercial lipid (Lipofectamine™2000), cationic dendrimer (Superfect™) and pPEG-trioxsalen amine (pPEGTX626).

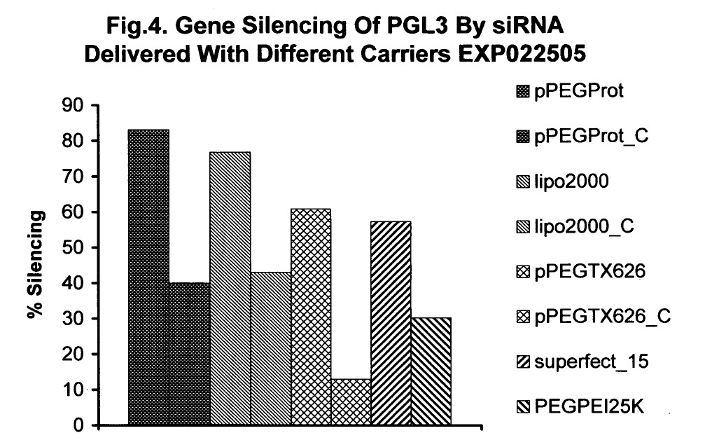

FIG. 5. Inhibition of condensation of DNA by pPEG-Hdz, pPEG-PEI2K (1020H), pPEGPEI.8K, and 1.2K, pPEG-Hdz cross-reacted with DMS (pPEG-DMS), linear PEG-PEI (1209B), pPEG-PEI25K, and hyperbranched pPEG dendrimer, in PEI condensation assay (EX121604). Dilution series of each sample were prepared in 50 ul water, complexed with 70 ul of 1 mg/ml DNA followed by 40 ul 1 mg/ml PEI, and turbidity read at A410.

FIG. 6. Transfection of COS7 cells with PGL-3 plasmid (Promega) encoding firefly luciferase, delivered with protamineX (67:1 mass ratio) or pPEGProt11105C (80:1 mass ratio), alone, with free hydroxychloroquine, or with hydroxychloroquine PEG conjugate (PEG-HQ) at 50 uM.

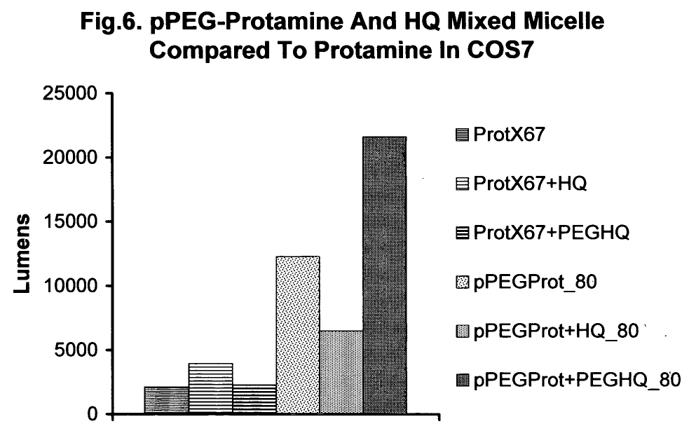

FIG. 7. Toxicity study of different carriers. Phosphorothioated DNA's, complexed with pPEG-PEI2K (pPEG1020H 17:1 N/P ratio), pPEG-Protamine (80:1 m.r. 50mMPEGHQ), cationic lipid (Lipo2000 3:1 m.r., Invitrogen Inc.), or cationic dendrimer (Superfect™, 30:1 m.r., Qiagen Inc.) were compared in human MCF-7 cells. Toxicity was assayed in triplicate, after 48 hour incubation, by MTT assay for mitochondrial dehydrogenase activity.

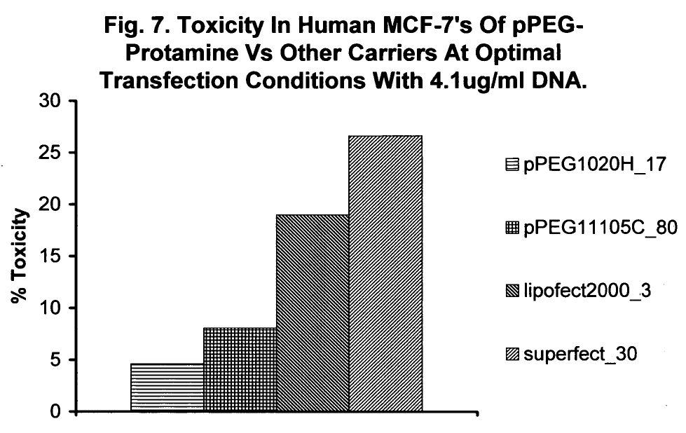

FIG. 8. MCF-7 cells (avg. 7000/well) were incubated 48 hours with pPEG-Protamine (m.r.100:1), cationic lipid (lipo2000 m.r. 3:1, Invitrogen), or cationic dendrimer (Superfect™, m.r. 30:1, Qiagen) complexed with 8.2 ug/ml (200 nM), single stranded, phophorothioated DNA, and then assayed for viability with MTT. The percentage toxicity was averaged from triplicates.

FIG. 9. Green monkey kidney (COS7) cells were transfected ex-vivo with pPEGProt:pGWz nano-particles encoding the firefly luciferase gene (EX111905). Post-transfection, the cells were transplanted into the mouse via tail vein injection. Bioluminescence from 760,000 COS7 cells expressing firefly luciferase gene, was detected in kidney, liver, and lung.

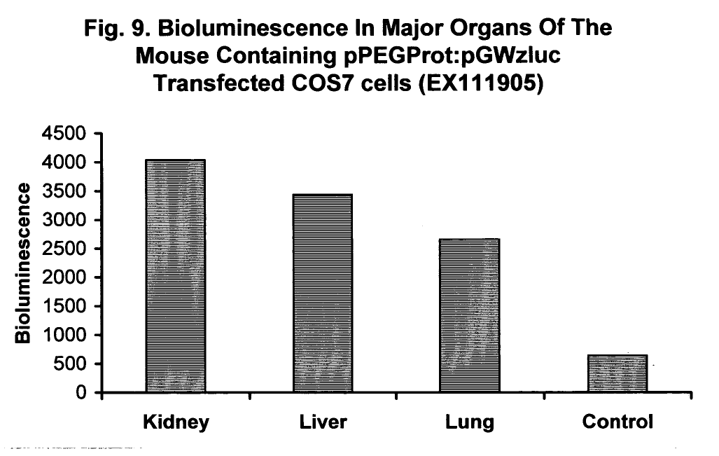

FIG. 10. Five different cell lines (MCF7 human breast cancer), COS7, 3T3, BHK21, (MRC5 human lung, primary) were transfected with pPEGPAM:pGWz nano-particles encoding the luciferase gene. The transfection efficiency of two carrier:DNA mass ratios was compared (50:1 and 70:1). Twenty four hours post transfection, the cells were analyzed for luciferase activity using reporter lysis buffer and luciferase assay reagent (Promega).

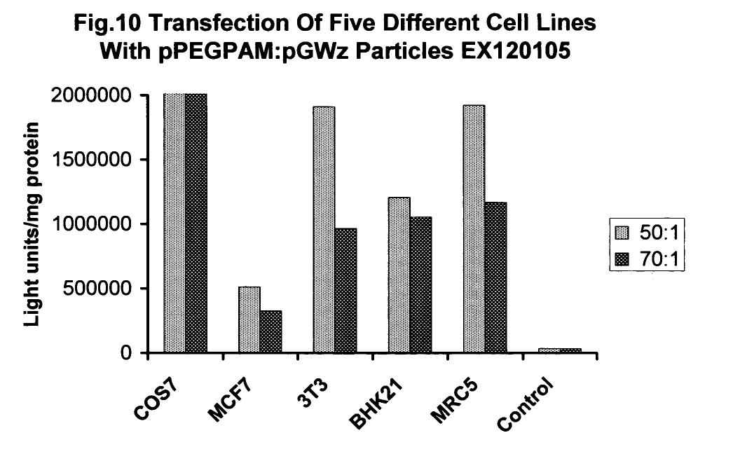

Bioluminescence was detected on the Synergy HT1 Biotek plate reader and normalized against total protein using brilliant blue G dye (Sigma-Aldrich).

FIG. 11. Shows the sequence specific inhibition on proliferating human breast cancer cells (MCF7) by pPEG-ODN conjugates (EX062604B, plate #2) prepared from trioxsalen coupling of G3139 anti-sense (AS, Genta Inc.) sequence or reverse sequence (Control).

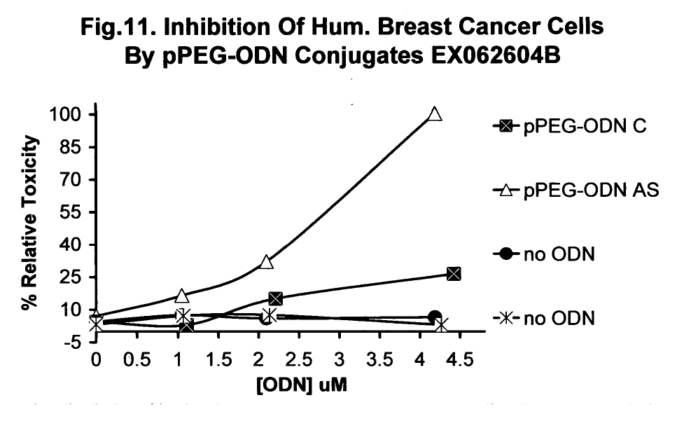

### DESCRIPTION OF THE PREFERRED EMBODIMENTS

The following definitions are disclosed for the purpose of defining the field and scope of the present invention.

Polynucleotide

The polynucleotide may be selected from a deoxyribonucleic acid (DNA), ribonucleic acid (RNA), oligodeoxynucleotide (ODN), phosphorothioate ODN (PS-ODN), diester ODN (PO-ODN), MOE-ODN, ribozyme, or siRNA. Polynucleotides may be single or double stranded DNA or RNA and include DNA and RNA hybrids thereof. Triple or quadruple stranded biologically active DNA's are also potentially useful in the invention. Also included are plasmid DNA and genes. Also included are DNAs or RNAs that have backbone modifications such as the phosphorothioates, methylphosphonate oligonucleotides, phosphoroselenate oligonucleotides, phosphotriester oligonucleotides, 2′-O-alkyl polynucleotides, and analogues. Also included are neutral analogues of DNA or RNA such as peptide nucleic acids or PNA's. Also included are synthetic and naturally derived DNA's and RNA's which are biologically active.

Bioactive Molecule

A “bioactive molecule” is a molecule which causes a measurable biological response in a cell or organism.

Oligonucleotide Drug

Oligonucleotides have shown promise in a number of different therapeutic applications.

Antisense oligodeoxynucleotides (or antisense ODN's) refers to short sequences of DNA that can Watson-Crick hybridize to a mRNA target and interfere with its function. Antisense ODN's such as G3139 (Genta Inc.) and c-raf among others, have been shown to down regulate the level of specific proteins within a cell by catalyzing the cleavage of the cellular mRNA that codes for the protein. This group also includes RNAi, or small interfering RNA's (siRNA's) which also hybridize with mRNA and facilitate mRNA cleavage. Small interfering RNA's have shown utility in selectively knocking out genes, and have important application in drug research. Interfering RNA's drugs have the potential of reducing required doses of chemotherapy agents and are showing promise as highly effective anticancer agents by themselves. ODN's have shown to be useful for causing single base pair substitutions in genes (Kren et al, 1998). RNAi's and ODN's have shown inhibition of viruses. Also included are antisense double stranded DNA or RNA. As defined herein, oligonucleotide drugs are nucleic acid sequences which can affect the function of a gene, protein, RNA, or DNA. Also included are nucleic acid sequences which inhibit the function of a bacteria or a virus.

Aptamer Drug

Aptamers are single stranded or double stranded nucleic acids which function by directly binding a target protein and interfering with its function (also referred to as DNA “decoy” drugs). Double stranded oligonucleotides have been shown to selectively interfere with the function of Taq polymerase. As defined herein, aptamer drug broadly encompasses polynucleotides used to interfere with the function of a protein by binding to said protein. Aptamer drugs include oligonucleotides which can bind to viral or bacterial proteins and inhibit the function of a virus or bacteria.

DNA Vaccine

Polynucleotide-based vaccines are defined herein as DNAs or RNAs which are used to elicit an immune response. For example, a plasmid DNA encoding a known antigen, such as a tumor associated antigen, can be transfected into a cell to immunize the cells against cancer [Irvine A. S. Efficient nonviral transfection of dendritic cells and their use for in vivo immunization, Nature Biotech. 18:1273-1278 (2000)]. “DNA vaccines” as they are known in the art, have the promise of providing long lasting immunity. However, effective carriers are needed for their effective distribution, stability in-vivo, etc.

Gene Replacement Therapy

As defined herein, gene replacement therapy encompasses the use of polynucleotides to add or replace missing genetic material or repair genetic material within a cell. These drugs have significant application to improving human health but also have significant potential for in-vivo and in-vitro research. In gene replacement, the polynucleotide might take the form of a viral plasmid DNA, in which the gene of interest has been cloned, and with the necessary promoter sequences, the plasmid would function to incorporate desired nuclear material within a cell nucleus. The polynucleotide might encode the necessary gene for a protein such as insulin. For example, gene replacement therapy has been shown to stimulate production of blood vessels. Gene replacement therapy has enormous potential for the treatment of inheritable diseases such as cystic fibrosis among others.

Linear PEG

As commonly used in the art, poly(ethylene) glycol generally refers to the linear form of poly(ethylene glycol) since these are the most common, commercially available PEG. Linear PEG can be represented by the formula OH—(CH2CH2O)n-OH (diol) or mPEG, CH3O—(CH2CH2O)nOH and these are commercially available from Sigma-Aldrich in a variety of molecular weights ranging from 3500 to 300000. Linear PEGs are available as monofunctional or bifunctional forms. PEG's may contain functional reactive groups at either end of the chain and can be homobifunctional (two identical reactive groups) or, heterobifunctional (two different reactive groups). For example, heterobifunctional PEG of the formula NH2-(CH2CH2O)nCOOH are commercially available and are useful for forming PEG derivatives.

Multi-Branched PEG (pPEG)

As defined herein, “multi-branched PEG” or “pendant PEG” (pPEG) comprises a polyoxyethylene glycol backbone with between 6 and 100 pendant arms extending from the core. The preferred polymer is defined as “multi-branched” such that the pendant arms extend away from the PEG backbone to form a comb-like structure, and each of the pendant arms extend from unique initiation points along the backbone. In the synthesis of multi-branched PEG, the pendant arm is grafted to the PEG backbone and may be suitably modified at its terminus such that the multi-branched PEG is multifunctional and provides multiple ligation sites along the PEG backbone. The pendant arms may comprise short alkyl chains or polyoxyethylene glycol chains or a combination thereof. Multi-branched PEG that are highly suitable in the present invention and herein incorporated for reference are available from the Sun Bio Inc. catalogue in a weight range of between 10K and 100K and available branching of 6, 8, 10, 12, 14, 16, 18, and 20 chains per mole. Multi-branched PEG's which have polyethylene oxide backbone substituted with polyalkylene oxide backbones such as polypropylene glycols, are also suitable in the present invention. Also suitable are multi-branched, comb-like PEG's obtained commercially which comprise a polyol core with between 6 and 100 PEG or alkyl chains, and may function similarly as polyethylene or polyalkylene glycol backbones in the present invention provided they have sufficient branching, stability, shielding, and amphiphilic qualities. With some modification, the amphiphilic quality of the multi-branched PEG can be controlled by adjusting the chain length and the terminal functional group. For example, in some of the compositions, the addition of strongly hydrophilic groups such as hydrazine or polyethyleneimine was shown to negate the micelle forming quality of the polymer by their excess hydrophilicity. Most preferred are multi-branched PEGs that produce micelle structure in water. Multifunctional PEGs which have one or more “endcapped” pendant arms are also included. Multi-branched PEG propionic acid of the formula PEG-OCH2CH2COOH is commercially available from SunBio Inc and is a homo-multifunctional PEG, such that it contains multiple arms terminated with reactive carbonyl sites that are suitable for derivatization. The multi-branched PEG propionic acid (pPEG) is highly preferred as a backbone polymer in the invention. Other multi-branched PEGs incorporated herein for reference, are available from the manufacturer which have the same backbone structure but have different terminal functional groups. Using methods available in the art, other multi-branched PEGs may be synthesized in which the carboxylic acid is substituted with an aldehyde, primary amine, sulfhydryl, epoxy, or activated NHS ester. As used herein, multi-branched PEG includes derivatives and modifications of pPEG, and can be represented as the formula PEG-[LX]y in which PEG is the poly(ethyleneoxide) backbone, L is a linker arm, and X is a functional group such as, COOH, COH, —NH2, —SH, —CH3, —OH, epoxy, —(NHS) etc. and y designates the number of branching or pendant arms and ranges from about 6 to 20. Also included are multi-branched PEG where the linker arm L is an alkyl chain (—CH2-)n and n is an integer from 1 to 12. Also included are derivatives in which L is a short chain grafted to a linear PEG chain to form pendant arrangement and L is represented as (-PEGX) or (alkyl-PEGX)— where X is a functional group. Also included are co-polymers of multi-branched PEGs (pPEG's) herein described and their derivatives. In the formula PEG-[LX]y, greater numbers of chains per mole are possible by cross-reacting the multi-branched PEG to form co-polymers of pPEG. The molecular weight of the multi-branched PEG polymer can range from 5000 to 400000 daltons.

In the formula PEG-[LX]y, the PEG backbone might also vary. Polymers which have multiple terminal hydroxyls can potentially serve as backbones in the synthesis of multi-branched PEG. In this case, the multi-branched PEG might be selected from a polyoxyethylated polyol, polyoxyethylated sacharide, a polypropylene glycol, or a polyalkylene glycol, where the number of branching arms (y) ranges from about 4 to 500 and X is a functional group.

Alternatively, the multi-branched PEG can be formed by synthesis of a PEG backbone utilizing glycidol and sec-BuLi to form a PEG core which has terminal hydroxyl groups extending from the backbone as shown below.

—CH2CH2[(CH2OH)sub1]-CH2CH2[(CH2OH)sub2]-CH2CH2[(CH2OH)subn]-

The hydroxyls are then reacted with ethylene oxide to form branching PEG chains [Peppas N. A. et al., Preparation and Properties of Poly(ethylene oxide) Star Polymers, J. App. Polymer. Science, 87:322-327 (2003)].

Since the amphiphilic quality is critical to formation of the nanoparticles of the invention, the hydrophilic and hydrophobic balance (HLB value) of the PEG polymer are important. Most preferred are multi-branched PEG of PEG-[LX]y which have equivalent or better micelle forming qualities as pPEG (multi-branched PEG propionic acid, SunBio Inc). For example, multi-branched PEG functionalized with propionic acid groups have a surfactant-like quality and have shown significant advantage over linear mPEG in forming the desired micelles of the invention. It can be appreciated to one skilled in the art that grafting hydrophobic or lipophilic groups, such as a lipid, to one or more of the chains would alter the amphiphilic quality of the multi-branched PEG. Likewise, grafting a highly polar, hydrophilic moiety to LX, such as a sulfate or carbonate group has a similar affect. Suitable hydrophobic groups may include cholesterol, fatty acid chains, vitamins, and peptides. Suitable hydrophilic groups may include phosphate, sulfate, or carbonate, among others. Surfactant like quality of the polymers in the present invention can be assayed by the critical micelle concentration in water as determined by light scattering.

Multi-Arm PEG

As defined herein, multi-arm PEGs are produced from a non-PEG backbone. Multi-arm PEG are synthesized by ethoxylation reaction of a polyol such as pentaerythritol (4 arm) or glycerol (3 or 8 arm) to produce multi-armed PEG's (see Harris, J. M. and S. Zalipsky, eds, Poly(ethylene glycol), Chemistry and Biological Applications, ACS Symposium Series 680, 1997). Examples of multi-arm PEGs produced from a backbone of sorbitol are commercially available from Sun Bio Inc. Multi-arm PEG's can be suitable in the present invention provided that they have a multiplicity of pendant arms and that they meet the other requirements of multi-functional PEG's previously described.

Star PEG

Star PEG are distinct from multi-branching PEG in that PEG chains are constructed from a single point, in a non-linear arrangement, such that polymer takes a more spherical configuration. Star PEG can be synthesized by reiterative synthetic propagation from a central core, utilizing ethylene oxide. In contrast, multi-branched PEG's are constructed so that the chains propagate along the backbone at multiple points to create a comb-like structure.

Backbone Polymer

Without limiting the invention to a specific mode of action, the “backbone polymer”, “core polymer” or “backbone” refers to the polymer that serves as the foundation polymer for synthesis of the copolymers of the present invention. In the synthesis of multi-functional PEG, the pendant or linker arms are grafted along the backbone polymer, such that the backbone polymer is common to the linker arms and to the optional groups that are grafted therewith. Other polymers may also be grafted onto the backbone polymer and extend from the backbone polymer in a pendant or branching configuration, such that the backbone serves as the common substructure to the other moieties in the polymer.

Spacer Arm

Spacer arm, linker arm, pendant arm, and branching arm; these terms are used synonymously to refer to the arm, represented symbolically as L or LX, that extends from the multi-branched PEG backbone. As defined herein, L (in some cases also represented by R) may comprise a PEG, an alkyl chain or a combination thereof which provide necessary chain flexibility and spacing between the backbone polymer and the other components which are optionally grafted to L. In addition, L may also comprise a plurality of functional groups which are useful for carrying out the invention. One or more L's may be endcapped in a synthetic step.

Functional Group

As used herein, a functional group is a group which may provide a ligation site during a synthetic step. The PEG chains might be suitably functionalized to provide for covalent bonding of various entities to the multibranched PEG chains. Functional groups that are useful in the present invention may include, hydroxyl, amine, carbonyl, aldehyde, ether, phosphate, sulfate, methyl, cyano, ketone, maleimide, succinimide, tosylate, homobifunctional cross-linker, heterobifunctional cross-linker, hydrazine, azide, alkylating agents, a leaving group, activated ester, thiol, epoxy, among others.

Cationic Polymers

Cationic polymers which may be useful in the present invention include PEI, and polyamido amines. Cationic polymers are defined generally as polymers which show capacity to bind or condense DNA. Cationic co-polymers can be prepared by covalently linking a PEI or cationic peptide with DSP, DMS, diepoxyalkane, or other amino reactive crosslinker to produce a cationic polymer of higher molecular weight.

Cationic Peptides

Oligo-arginines, GALA, KALA, high molecular weight protamine (HMWP), low molecular weight protamine (LMWP), protamine from herring or salmon sperm, and polylysine.

Targeting Moiety

As defined herein, a molecule which is specific to a cell surface receptor or ligand, such as an antibody, antibody fragment, peptide, viral peptide, vitamin, folic acid, steroid, or transferrin. Viral peptide fragments such as TAT are known to be effective at promoting uptake of liposomes into cells and across cell membranes (Torchilin et al, Pub Med Central, 2001). The influenza HA2 peptide, Polymyxin B, is also described [D. Deshpande, D. Toledo-Velasquez, D. Thakkar, W. Liang, Y. Rojanasakul, Pharm Res. 13:57-61(1996)]. Other cell targeting moieties which may have utility in the present invention include the cell targeting peptide, RGD [(Schiffelers, R. M et al. Nuc. Acid. Res. 32:e149 (2004)]. Targeting moieties are preferably coupled to the backbone polymer, via the pendant or branching arm. Coupling of the polynucleotide to the targeting peptide is less preferred since it potentially impedes function of the peptide [Antopolsky M., et al, Peptide-oligonucleotide phosphorothioate conjugates with membrane translocation and nuclear localization properties, Bioconj. Chem. 10: 598-606 (1999)].

Membrane Permeabilizing Agent (MPA)

Certain molecular entities can act as agents to promote uptake of polynucleotides across cellular membranes or escape from intracellular compartments. Incorporation of MPA's into the micelle particle may function to enhance uptake and or endosomal release. Some MPA's such as chloroquine, have shown unexpected benefits and enhanced performance in the present invention only when conjugated within the micelle. Conjugation is effective, provided that the MPA is exposed at the surface of the micelle, such that it may be active. MPA's which may have utility in the invention include chloroquine, hydroxychloroquine, primaquine, and their derivatives and analogs.

Polyelectrolyte Complex

The polyelectrolyte complex, lipoplex, or polyplex is a particle typically formed at nitrogen/phosphate (N/P) ratios that are sufficient to condense the polynucleotide and produce highly turbid, flocculated solutions with DNA. Polyelectrolyte complex particles are unshielded, and have positive surface charge that promotes aggregation and cellular uptake. Increasing N/P ratios in these particles typically leads to higher zeta potential, and smaller hydrodynamic radius. Polyelectrolyte complexes have been described as torroidal in shape. Cationic backbone polymers which have been used in the formation of polyelectrolyte complex with DNA include PEI's, cationic dendrimers, cationic peptides (protamine), oligoarginines, fusogenic peptides (ie KALA), cationic lipids (DOTMA, DOTAP), polylysines, and polylysine galactose derivatives.

Cationic Delivery System

A polynucleotide delivery system that employs the use of cationic agents to cause a condensation of the DNA or polynucleotide. In such systems, sufficient cationic agent is added to the polynucleotide to form polyelectrolyte particles with positive surface charge, typically at N/P ratio of 2:1 to 20:1. The condensation of polynucleotides in cationic systems typically results in turbidity, flocculation, or sedimentation at N/P ratio of 2:1 to 20:1. Sedimentation is significant in these systems, particularly in ionic solutions and solutions containing serum proteins. Polyelectrolyte sediments are in some cases desired in these systems and have been described as being useful for coating the cells and facilitating transfection. By their nature, cationic delivery systems employ the use of unshielded particles, utilizing positive surface charge to promote binding to negatively charged cell surfaces. Cationic systems are generally systems which do not protect the polynucleotide from condensation at final N/P ratios in ionic solution of between 2:1 and 20:1.

Shielded Micelle

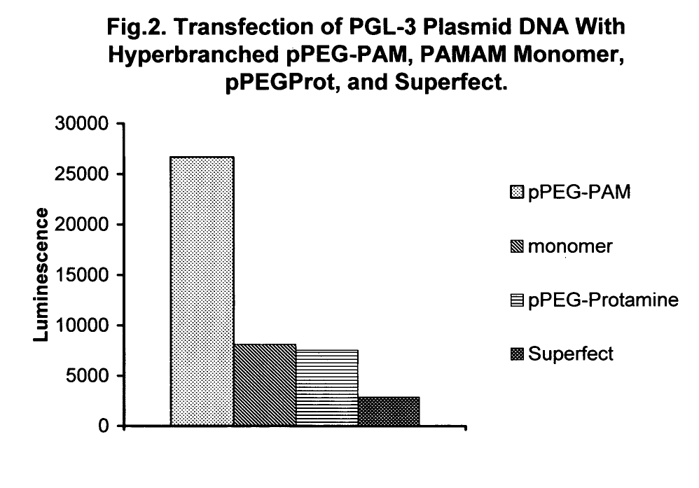

The incorporation of a polynucleotide into the shielded micelle produces a unique particle, a shielded polynucleotide particle, which sterically isolates the polynucleotide from the outside. The term “shielded” refers to the unique capacity of the micelles to resist condensation by polycations at high N/P ratios, typically between 4:1 and 20:1 and is measured by the PEI condensation assay. More broadly, the terms “shielded” or “shielding” refer to the capacity of the polymer compositions to reduce the condensability of the polynucleotide with a cationic agent as determined in the PEI condensation assay by a reduction in turbidity at between 2.5:1 and 4:1 N/P ratio. In more preferred embodiments, the polymer compositions have the capacity to produce an OD reading at 410 nm of between 0.3 units and 0.01 units at between about 1.5 to 0.5 PEG/DNA mass ratio (see FIG. 2, 3) where the baseline OD is typically 0.05. The OD range may slightly vary at the baseline, but in general the shielded micelle compositions produce a sharp drop in turbidity starting at about 0.25 PEG/DNA mass ratio, which levels off at between about 1.0 and 2.5 PEG/DNA mass ratio (m.r.). The polymer compositions have reduced the turbidity of DNA by an order of magnitude lower than PEI alone at about 0.5 DNA/pPEG mass ratio and 4:1 N/P ratio. The capacity for shielding DNA is apparently a property of the multi-functional PEGs, as pPEG generally have shown higher shielding of charge than linear PEGs conjugated to cationic agents, such as PEI's. However, shielding is also related to how the polymer is synthesized since some isomers were found to be less efficient at protecting the polynucleotide against condensation. When comparing the suitability of the multi-functional PEG polymers and structures, the more preferred are polymers which produce the greatest isolation of the polynucleotide charge, greater transfection efficiency, and show protection from condensation by PEI.

The mass ratio required for suppression of condensation is a means for determining structure of the polymers and steric stability of the micelles but may also predict transfection efficiency. Mixed micelle compositions which potentially provide similar reduction in condensation are also preferred. Without limiting the invention to a specific mode of action, it is hypothesized that the shielding effect is likely due to steric isolation of the cationic charge from the anionic charge of the DNA. It has also been discovered that shielded particles of the invention are sterically tight enough to prevent intercalation of the DNA by small molecules. Surprisingly, it was discovered that incorporation of the polynucleotide into the micelle particle can be accomplished by covalent or non-covalent association with the amphiphilic, multifunctional PEG. The depression of turbidity is a measure of the binding affinity of the polymer for the polynucleotide and so is dependent on covalent or non-covalent bonding. For example, conjugated pPEG-ODN micelles were shown to reduce condensation of the phosphorothioated oligonucleotide by PEI. When the DNA is bound to the multi-functional PEG by non-covalent means, the isolation of charge from the outside by the micelle structure is believed to reduce the toxicity of the carrier. Isolation of the anionic charge of the DNA from the outside is also believed to facilitate uptake into the cell, escape from endosomes, and intracellular trafficking.

Transfection Assay

The suitability of multi-branched PEG polymer in the uptake and transport of PEG-DNA particles is determined empirically by their transfection efficiency. Transfection efficiency of a micelle system is assayed by transfecting a low concentration of PGL3 control vector (Promega Inc) into COS7 cells and quantitating luminescence. More efficient uptake of the plasmid into cells over time results in higher luminescence signal. The transfection efficiency of the nanoparticles of the present invention have been found to vary based on the functional group and on the ordered structure of the micelle.

PEI Condensation Assay

In the presence of PEI, DNA will normally condense to form highly turbid solutions.

However, when DNA is incorporated into the multi-branched PEG micelles, turbidity is significantly reduced. The reduction in turbidity is associated with the steric interference (shielding effect) of the bulky, multi-functional PEG on the surface of the polyplex particle. The PEI condensation assay is therefore a spectrometric method for looking at the micelle forming capacity of the polymer compositions with DNA and making comparisons with other systems. For example, at a 4:1 nitrogen to phosphate ratio, PEG/DNA micelles prepared with multi-branched PEG of y=12 show greater clarity at 410 nm than equivalent micelles prepared with bi-functional PEG. The PEI condensation is typically assayed in a 96 well polystyrene plate by combining 50 ul of the sample with 70 ul of 1 mg/ml DNA, followed by 36 ul of 1 mg/ml PEI25K pH7. Sample absorbance is read at 410 nm on a Synergy HT1 microplate reader.

Clear Dispersion

A solution that is substantially free of turbidity or flocculation. As defined herein, a solution that produces an OD reading at 410 nm of 0.1 or less in 0.2 ml (using a Falcon™, UV-clear plate and Synergy HT1 plate reader). The shielded micelles herein produce clear dispersions at typically less than 0.05 OD when combined with the polynucleotide.

Molar Ratio

Molar ratio is used to refer to conventional mole to mole ratio. For example, in formula II, the molar ratio of nitrogen rich moiety (N) to multi-branched PEG can range between 0.01:1 and 200:1. In other words, in the conjugation of protamine to the multi-branched PEG, the moles of protamine can comprise a molar ratio of 1 to 100 (0.01:1) to as high as 200 to 1 (200:1), for each multi-branched PEG. In the case that the mole ratio of protamine to pPEG is (0.01:1) or 1 to 100, it is understood that more than one multibranched PEG can conjugate to a single protamine.

Hyperbranched PEG Polymers

These polymers are a unique class of dendritic polymer, distinct from dendrimers. In hyperbranched polymers the dendritic chains branch from multiple locations along the core polymer backbone to form a comb-like, pendant shaped polymer (C. Gao, D. Yan Hyperbranched polymers: from synthesis to applications, Prog. Polym. Sci 29:187 (2004)).

Biocompatible, Non-Alkylene Oxide Polymers

Other biocompatible polymers that can be suitably modified to provide branching, functionalized chains include N-(2-hydroxypropyl)methacrylamide (HPMA) and poly(lactide-co-glycolide (PLGA).

Dendrimers

Dendrimers are a class of dendritic polymer characterized by highly structured dendritic branches extending from a central core to form a rigid, nano-sphere (C. Gao, D. Yan Hyperbranched polymers: from synthesis to applications, Prog. Polym. Sci 29:187 (2004). Polyamido amine dendrimers (PAMAM) are viscous liquids, slightly insoluble in water, but highly soluble in 100% methanol (Dendritech Inc).

Intercalators

Nitrogen containing, heteroaromatic molecules which interact with DNA by intercalation are suitable in the invention for providing non-covalent associations between the micelle forming polymers and the polynucleotide. Potentially useful are the anthracene derivatives, anthraquinone antibiotics and derivatives, anthracene diones, phenanthrenes, psoralen amine, and trioxsalen amines. Minor groove binders such as DAPI and Hoechst dyes are a separate class of DNA binding agents and can have similar utility as DNA intercalators in the invention.

### The Composition

This invention is a new class of compositions for delivering polynucleotides across cellular membranes, comprising a shielded micelle polynucleotide particle, further comprising an amphiphilic, multifunctional polymer which is either covalently or non-covalently attached to the polynucleotide. When the amphiphilic polymer is combined with the polynucleotide, it forms a shielded micelle particle with the polynucleotide in which the negative charge of the polynucleotide is sterically shielded from the outside. The shielding of the polynucleotide by the sterically bulky, amphiphilic polymer is believed to facilitate cellular endocytocis and subsequent escape from endosomal membranes.

One of the principal advantages of the composition over prior art is that high rates of transfection can be achieved without forming a sedimentation or precipitation of the polynucleotide. Depending on the ratio of polymer to polynucleotide employed, the compositions can be prepared with the polynucleotide to produce clear dispersions. The particles are shown to be highly stable and resist condensation by PEI at nitrogen to phosphate (N/P) ratios of between 4:1 and 20:1. The enhanced solubility of the present composition increases the bioavailability of the polynucleotide for cellular uptake.

In the preparation of the shielded micelle particles, multifunctional PEG is a preferred backbone polymer and has many desirable properties including high water solubility, biocompatibility, and efficient polynucleotide loading. Loading of polynucleotides within the micelle particles is enhanced with the use of multifunctional PEG, since multiple polynucleotides can be covalently attached to the PEG via pendant or branching arms. High polynucleotide loading can also be achieved in the micelle when the pendant or branching arms contain a nitrogen rich moiety. Micelles created by non-covalent association of multi-functional PEG with the polynucleotide have also been found to be highly efficient carriers, with greater shielding and transfection efficiency than other polymers. The shielded micelles have negligible toxicity at the highest concentrations tested, making these carriers suitable for pharmaceutical application.

The following describe the micellular component polymers or amphiphilic, multifunctional PEGs that comprise the shielded micelle composition. The preferred amphiphilic polymer carrier may be represented by PEG[LX]yQ (Formula I) where PEG is the amphiphilic, multi-branched PEG, L is a spacer arm, X is a functional group covalently bound to L, y is the number of branching arms, and Q is the polynucleotide.

Formula I PEG[LX]yQ

In one embodiment of the invention, the composition comprises multi-branched PEG of 2000 to 100000 daltons, preferably containing between about 4 and 500 pendant arms, more preferably between about 6 and 200, and one or more polynucleotides which are incorporated in the micelle by covalent attachment to the backbone polymer. The pendant or spacer arms provide multiple ligation sites for covalent bonding of polynucleotides, such that multiple polynucleotides may be grafted to a single amphiphilic PEG backbone. In this case the formula is PEG[LX]yQ in which the backbone polymer is PEG, the linker or spacer arm is L, X is a functional group covalently bound to L, y is the number of branching arms, and the polynucleotide is Q. In this case X is an appropriate functional group that may be used to provide a covalent linkage between the linker arm and Q. Operatively, a proportion of X may also be selected from a methyl or a hydroxyl such that they are non-reactive but contribute structurally to the polymer. Conjugation to “LX” refers to conjugation to the branching arm but may refer to conjugation to the group X or to a functional group on L depending on the structure of LX and the synthetic requirements. In some cases, particularly when X is not suitably reactive, the branching arm L might comprise an additional site of reactivity in addition to X that can serve as a site for covalently grafting other components in the composition. The backbone polymer may contain between 4 to 500 mole ratio of linker arm L per mole of polymer and more preferably between 6 and 100. The polynucleotide can be covalently attached to the backbone polymer via the linker using an ester, peptide, amide, hydrazone, thiol, ether, or other suitable covalent linkage between X and Q that is stable at physiological pH. The polynucleotide molar ratio to the polymer is preferred to be between 1:1 and 400:1 per mol of polymer. In this composition it is preferred that the polynucleotides Q, will be covalently attached to the linker arm via the 3′ or 5′ end of the polynucleotide to avoid the potential for steric hindrance during hybridization of the polynucleotide to a target. The polynucleotide may also be grafted to the linker arm via the backbone of the polynucleotide. This composition may be particularly useful for delivery of polynucleotides which do not have a negative charge or for neutral polynucleotides such as peptide nucleic acids (PNA's).

The Backbone Polymer (PEG)

In the formation of shielded micelles, it is preferred that the backbone polymer is a multi-branched, multi-functional PEG of molecular weight between 5000 and 500000 daltons, comprising between 6 and 500 branching chains, and is amphiphilic. Highly preferred are multifunctional PEG's that exhibit a hydrophobic and hydrophilic side and form micellular structure in water. The multi-branched pPEG available from Sun Bio Inc. of 20,000-70,000 molecular weight, 12-20 terminal propionic acids groups, and 12-20 branches are highly preferred in forming shielded micelles of the invention. The multi-functional PEG is preferably biocompatible, structurally stable in physiological saline pH7, substantially non-cytotoxic in cell culture and suitable for use in-vitro or in-vivo. In yet another embodiment, the branching or pendant arms on the multifunctional PEG backbone are preferably functionalized with hydroxyl, carbonyl, amine, amide, epoxy, activated ester, or other suitable reactive species, to provide ligation sites. The multifunctional PEG may further comprise one or more arms which are encapped such that they provide structural integrity to the polymer but are not necessarily suitable for ligation or association with the polynucleotide.

Spacer Arm L

The spacer arm L is grafted to the backbone polymer to form a branching or pendant configuration by methods of synthesis known in the art. Each arm can be functionalized with a reactive species to provide a ligation site for covalent bonding of the polynucleotide. For example, the amphiphilic PEG may be selected from pendant PEG propionic acid of 20K daltons with 12-15 moles of spacer per mole of polymer such that each L has a terminal carboxilic acid group. It is preferred that the spacer (or pendant) arm is of suitable length to reduce steric hinderance and hybridization of the polynucleotide drug to a target. The polynucleotide can be covalently coupled to the spacer arms L via a peptide, ester, hydrazone, thiol, ether, amide, or other suitable covalent bond that is stable at physiological pH. For example, the covalent coupling between the polynucleotide and the spacer or linker arm L can be provided by functionalizing the polynucleotide at the 5′ or 3′ end with a primary amine and reacting the functionalized polynucleotide with an activated NHS ester, a heterobifunctional linker (SPDP), an aldehyde, or other amino reactive group on the L, to form a covalent bond. The polynucleotide may also be covalently attached to the spacer arm via a backbone extension. For example, a reactive species may be incorporated into the backbone of the polynucleotide via a spacer molecule at one or more non-bridging oxygens and subsequently grafted to L by NHS ester, a heterobifunctional linker (SPDP), an aldehyde, or other suitable reactive group to form a covalent bond.

The polynucleotide (Q) may be single stranded or double stranded. In the case that it is double stranded, it is preferred that only one of the strands is covalently linked to the pendant PEG polymer. In some applications, such as antisense therapy or siRNA's, this would be useful to provide for the hybridization of the antisense strand to a sense strand that is covalently coupled to the pendant PEG polymer. The active antisense strand can then be non-covalently associated with the complimentary sense strand. This configuration is useful for reducing degradation of the polynucleotide since double stranded polynucleotides are more stable in plasma. Double stranded polynucleotides conjugated to the amphiphilic polymer also may exhibit improved hybridization to target proteins in the cell, and would therefore be highly useful in the composition.

Formula II PEG[LX]yNQ

In another embodiment of the invention, the polynucleotide is incorporated into the shielded micelle particle by non-covalent association with one or more pendant arms containing one or more nitrogens. In this case, N is a moiety which contains nitrogens and is useful for non-covalent association with the polynucleotide (Q). It has been discovered that association of the polynucleotide with nitrogens on the pendant arms is sufficient to facilitate formation of shielded micelle particles, provided that the nitrogens are accessible to the polynucleotide. Hydrazine or 1,6 hexane diamine covalently attached at the terminal end of the L, have been found to provide excellent association with polynucleotides. Other amino compounds would likely be suitable for association, particularly ones that contain primary nitrogens. Nitrogen containing moieties that are potentially suitable include hydrazine, diamines, triamines, quaternary amines, amino acids among others. Also included are nitrogen containing derivatives of anthracenes, acridines, anthraquinones, anthracene diones, phenanthrenes, phenanthrenequinones, DNA intercalators, and minor groove binders, which bind with DNA non-covalently. For example, Doxorubicin, an anthraquinone antibiotic, was intercalated with herring sperm DNA and its amino group was conjugated to the multi-branched pPEG-aldehyde via a peptide bond. In PEG[LX]yNQ the amino containing groups can be incorporated into [LX] by substitution of N for X, or by grafting directly to X, such that the nitrogens are accessible to associate with Q. For example, in the formula, X can be a carboxylic acid group and N is a hydrazine that is crosslinked with X via catalysis by N-hydroxy succinimide and 1-ethyl-3-(3-dimethylaminopropyl)carbodiimide hydrochloride (EDC). The spacer arm L may comprise between 1:1 and 400:1 mol ratio to the polymer. The combination of PEG[LX]yN with Q in water are shown to produce shielded micelle particles. Stability of the micelle particle of the present invention is increased by factors such as steric compatibility between chains and by formation energy of the micelle. Multifunctional PEG's that are amphiphilic are therefore highly preferred in the present invention. For example, it has been discovered that a pendant PEG of 20K Daltons containing 12-15 pendant carbonyl groups reacted with between 1 and 12 hydrazines per mol, can form stable micelles with polynucleotide, and shield the polynucleotide from condensation in the presence of PEI at high N/P ratios. Other examples are provided that show that pendant PEG-hexanediamine can also provide suitable association with the polynucleotide to form shielded micelles. These compositions were surprisingly effective at transfection, since hydrazine and hexanediamine by themselves, are not known to transfect polynucleotides. The selection of synthetic conditions should be appropriate to give micellular polymer units that have nitrogens which are highly available to associate with the polynucleotide. It has also been observed that certain isomers can be prepared of pendant PEG hydrazine that do not form shielded micelle structure, possibly due to steric hinderance and or unavailability of the nitrogens to associate with the polynucleotide. Structural and or steric considerations are therefore important in synthesis of the optimal micellular components.

In the formula PEG[LX]yNQ, the backbone polymer PEG is preferably selected from the same class as in formula I. It is most preferred that the backbone polymer is selected from amphiphilic, multi-branched PEG of between 2000 and 1000000 daltons and contain between 1 to 400 moles of L per mol of polymer.

Spacer Arm LX—

In PEG[LX]yNQ, the spacer arm L is grafted covalently to the amphiphilic backbone by methods suitable in the art. In one example, the amphiphilic polymer, PEG, is multi-branched PEG propionic acid (pPEG) of 20K Daltons and contains 12-15 moles of L per mole PEG backbone. In this case, LX is a short, three carbon alkyl chain terminated with a carboxylic acid. In other preferred embodiments, each LX can be of the general formula CH2CH2COX, where X is selected from NH2, —NNH, —NCH2subnNH— (diamino), or alkylcarbazate —CH2(subn)-NNH. The pendant LX may have a general formula such as (CH2subnCO—X) in which n is between 1 and 20 and X is selected from NH2, —NNH, —NCH2subnNH— (diamino), or alkylcarbazate —CH2(subn)-NNH, —CON—CHsubnCON. L may further comprise a PEG, poly(alkylene) oxide, or alkyl chain or a combination thereof and may further comprise one or more functionalities which are grafted to the amino compounds indicated to provide nitrogens which can associate with the polynucleotide. The LX must also be sterically compatible, such that the micelle formation is not inhibited.

In formula II PEG[LX]yNQ the invention can potentially gain advantage by incorporating more nitrogens at the terminal ends of the spacer arms to improve binding and association with the polynucleotide. A nitrogen rich moiety (N) may be grafted to the amphiphilic PEG backbone via the spacer arm L by a carbonyl, activated ester, epoxy, NHS ester, or other suitable reactive species known in the art. In this embodiment, it is most preferred to sterically limit the molecular size of the nitrogen rich moiety and LX such that the nitrogens can provide necessary and sufficient association with the polynucleotide and be sufficiently shielded by the micelle from the outside. In this case the invention still operates as a shielded micelle but the binding strength of the multi-branched PEG is increased substantially toward the polynucleotide Q. Covalent bonding of nitrogen rich moieties such as linear or branched PEI's such as —CH2CH2-N(—CH2CH2Nsubn)-CH2] or (CH2CH2N-)subn to the pendant arms are demonstrated to provide excellent results in the present invention. Examples of multi-branched, amphiphilic PEG-PEI have been synthesized by the inventor and are disclosed. The amphiphilic PEG-PEI's in the present invention were found to have unexpected properties. It was surprising to discover that multi-branched PEG-PEI formed micelles that produce clear dispersions and prevented condensation of the polynucleotide at N/P ratios of 3:1, since normally PEI causes significant opacity at these ratios. The grafting of PEI to the pendant PEG provides excellent binding characteristics with the polynucleotide without the disadvantages normally associated with PEI. Furthermore, the toxicity of the resulting pendant PEG-PEI polymer is substantially less than PEI alone. In compositions that utilize nitrogen-rich, cationic polymers, it is highly preferred that the polynucleotide is non-covalently bonded to the micelle forming polymers.

Naturally occurring peptides which associate with DNA, such as protamine and the like, are potentially useful in forming the shielded micelles of the present invention when conjugated to the multi-functional PEG backbone. Protamines and other DNA binding peptides have advantages since they are derived from natural sources and typically have greater biocompatibility than PEI's. Synthetic cationic peptides, composed of arginine, as well as synthetic protamines, are also contemplated as being useful in the invention. Low molecular weight protamines, which can be prepared by column purification, or through enzymatic digestion of native protamine, can also be useful in the present invention [Singh et al, Low molecular weight protamine: a potential nontoxic heparin antagonist, Thromb Res. 94:53-61 (1999)]. Furthermore, protamines have already been approved for clinical use in cardiovascular surgery to neutralize heparin. For example, between 1 and 15 protamines can be incorporated onto a pendant amphiphilic PEG backbone of 20,000 (20K) daltons. Amphiphilic PEG polymers of higher weight 20K to 500K could potentially anchor more DNA associating moieties to provide more amino groups for association with larger polynucleotides and for greater shielding. When preparing the compositions it is preferred that the molar proportion of backbone polymer and spacer be in the range of 1:1 to 1:400, and the spacer arm be sterically limited such that the amphiphilic polymer will substantially incorporate the polynucleotide within the shielded micelle and produce clear dispersions with the polynucleotide.

Hyperbranched PEG polymers, comprising one or more pendant polyamido amine chains, have been synthesized by the inventor and found to be highly effective gene carriers. In hyperbranched PEG polymers, the dendrimer can be prepared by reiterative polymerization of multi-branched PEG amine core with for example, 2-acryloylamino-ethyl)-carbamic acid tert-butyl ester or N-cyanomethyl-acrylamide. The dendritic chains can be grown by reiterative steps to form successive generations with the desired branching and terminal amino groups. The hyperbranched polymers of the present invention differ from star shaped dendrimers in that the branching chains are initiated from the backbone from a plurality of junctions to form a comb-like, pendant polymer. Hyperbranched PEG polymers are shown in the disclosure to have significantly greater transfection efficiency over classic star-shaped dendrimers such as PAMAM. The higher transfection efficiency of the hyperbranched polyamido PEG polymers (pPEGPAM) is likely due to their formation of micelles with the polynucleotide as opposed to the condensed, polyplex particles obtained with the PAMAM. In the formula PEG[LX]yNQ the backbone is a multi-branched PEG such as pPEG and N are the dendritic, polyamido amines conjugated to LX and Q is non-covalently bound to N. In formula II, X can be a suitable initiator core known in the art, further comprising a reactive residue such as an amine, diamine, carboxylic acid, among others. Dendrimer polymers which are suitable for synthesis of hyperbranched co-polymers in the present invention are available commercially from Dendritech Inc as generation 1 to generation 10. Alternatively, a hyperbranched, polyamidoamine PEG can be constructed from the pPEG backbone by building dendritic polymers along the backbone in reiterative steps. Branching in the hyperbranched polymer is controlled so that N can range from a generation 1 to generation 10.

Mixed micelle formulations, comprising one or more additional components, are also anticipated as advantageous in the present composition. In some aspects of the invention, it has been discovered that linear PEG can be incorporated into the multifunctional PEG-PEI micelles to form a mixed micelle at mass ratio of between 1/100:1 and 10:1 to enhance performance of the present invention. It is probable that linear PEG contributes to the micelle structure as a neutral component since it is shown to reduce toxicity of the multifunctional PEG-PEI micelle. Furthermore, conjugation of chloroquine to linear PEG, a membrane permeabilizing agent, was found to substantially enhance the performance of the shielded micelle carrier over chloroquine alone. The chloroquine-PEG conjugate was not effective at enhancing transfection performance in conjunction with cationic agents, whereas free chloroquine in some cases doubled performance. Other chloroquine conjugates where shown to reduce effectiveness of the carrier. Some of the chloroquine conjugates were found to be less effective or inhibitory in the present invention, possibly due to disruption of the micelle structure, inactivation of the active agent, or both. In some cases the membrane permeabilizing agent may have been inaccessible and demonstrate the importance of micelle structure in efficacy of the carrier. Likewise, free chloroquine was found to inhibit function of micelles in some experiments. The enhanced performance of chloroquine-PEG conjugate was more significant when used in conjunction with the pendant PEG micelles, suggesting that the chloroquin-PEG enhances the function of the micelle rather than acting solely as a membrane disrupting agent. Other molecular entities which provide similar membrane permeabilizing function to chloroquine, may enhance performance of the shielded micelle carrier when conjugated to PEG and incorporated in the micelles of the present invention. These include chloroquine derivatives, primaquine, and primaquine analogues which are defined herein as membrane permeabilizing agents or MPA's.

In the formation of mixed micelles, similar utility can also be gained from inclusion of pegalated surfactants known in the art, with the multi-functional PEG micelles. Pegalated, pharmaceutical-grade surfactants such as the pluronics series (Sigma-Aldrich catalogue), can be combined at appropriate mass ratio with the pPEG micelles in order to further enhance stability and shielding of the multi-functional PEG micelles. More preferred are linear PEG's surfactants with a hydrophilic and hydrophobic end, that are further modified to comprise a nitrogen containing moiety, preferably containing between one and ten nitrogens which are capable of non-covalent association with the polynucleotide. For example, the linear PEG can comprise a suitable anthracene, acridine, anthraquinone, or phenanthrone derivative which has available amino groups or nitrogens for intercalation of the polynucleotide.

It can be appreciated that the invention combines multiple elements which by themselves are not substantially active or effective vectors but when combined in the modes described, form potent transfection agents. For example, the spacer arm may be functionalized with a compound such as a hydrazine or hexanediamine, compounds that are not suitable for tranfection of polynucleotides by themselves. However, when the nitrogen containing moieties are grafted to the amphiphilic polymer to form shielded micelles, they become potent agents for facilitating the translocation of polynucleotides into the cytoplasm of a cell. Likewise, in Formula I the conjugation of a polynucleotide to a multifunctional PEG can facilitate delivery of the polynucleotide across a cell membrane by itself, without the necessity of using any other transfection agents. In cell culture, the efficacy of the polynucleotide against tumor cells is substantially increased when combined with the micelle forming agents of the present invention. Furthermore, the invention can be used to form clear dispersions with the polynucleotide in the presence of DNA condensing agents at high N/P ratio of 3:1 to about 40:1, currently not achievable in the prior art.

In yet another embodiment, the polynucleotide can be covalently bound to the backbone polymer using a covalent attachment site that is not at the 3′ or 5′ end. For example, the incorporation of a primary amine or aldehyde residue by phosphoramidate chemistry into one or more nucleotide bases would provide a potential ligation site. Examples provided in the present disclosure demonstrate the principal by showing that a primary amine can be introduced into the polynucleotide via trioxsalen amine (a DNA intercalator) and subsequently grafted to the multifunctional PEG after cross-linking with glutaraldehyde. The example shows the feasibility of covalent bonding the spacer arm to a nucleotide at a location within the PN chain.

It was discovered that structural factors influence the effectiveness of the polymers in forming shielded micelles with the polynucleotide and must be considered in design of the carrier. For example, one of the compositions, pendant PEG hydrazine, can be prepared as several different isomers but only one isomer is highly effective at forming shielded micelles with a polynucleotide.

Similar results have been observed in experiments with pPEG-PEI's. It is important that the PEI's selected and grafted to the pendant PEG must not be excessively bulky to potentially interfere with the formation of the shielded micelle with the polynucleotide. If the PEI is sterically too large in proportion to the PEG the subsequent micelle structure may be inhibited and the toxicity substantially increased. Some of the PEG-PEI's were found to be ineffective at causing transfection of PGI3 plasmid in COS-7 cells and others were found to be highly effective. Some of the micelles where found to be more toxic due to the bulkiness of the PEI that was grafted to the polymer. In one embodiment, the PEI was conjugated directly to the pendant arm of the amphiphilic PEG and was discovered to have substantial binding and subsequent shielding of the polynucleotide at very low molar PEG/phosphate ratio. Pegalation of a PEI with a linear, bifunctional PEG was found to produce highly turbid solutions with a polynucleotide. Polymers prepared by conjugation of linear PEG to protamine were found to be significantly more toxic than pPEG-protamines in COS7 cells (EX030705). It can therefore be appreciated that the unique qualities of the shielded micelle compositions stem from the rational design of the micelle carriers to produce the desired properties of low toxicity, high transfection rates, solubility, and bioavailability.

Without limiting the invention in any way, it is possible that a multifunctional PEG can be grafted to several PEI's or, a single PEI can also be grafted to multiple pendant PEG's. If multiple pendant or branching PEG's are grafted to a single PEI, then the ratio in the formula will differ slightly. The molar ratio of PEI's to the PEG can range from between 1:1 and 1:400, respectively, depending on the size of the PEI. If PEI's serves as the template for attachment of multiple, multifunctional PEG's the resulting particles can fulfill the requirement of the invention, provided that they can form clear dispersions and facilitate translocation of the polynucleotide.

In other embodiments, antisense ODN was conjugated to the amphiphilic PEG via pendant propionic acid groups. It was discovered that the amphiphilic PEG-ODN conjugate (pPEG-ODN) was effective at eliciting an antisense response in MCF-7 cells. This was surprising for a number of reasons. 1) The ODN was delivered across the cellular membrane using a biologically inert molecule (PEG) and without using a cationic delivery system or other known transfection agent. 2) The ODN escaped the endosome and was transported to the nucleus, and 3) The ODN was effective at Watson Crick hybridization to a mRNA target within the nucleus, despite being conjugated to a bulky polymer.

In subsequent embodiments it was discovered that when amphiphilic PEG hydrazine was combined with antisense ODN, this composition also produced an antisense response in MCF-7 cells. This was surprising, since the ODN was not conjugated to the PEG, nor was the ODN charge neutralized, as required in cationic delivery systems.

In another embodiment, trioxsalen amine (TX) was used to conjugate antisense ODN to the amphiphilic PEG via intercalation of trioxsalen within a thymidine rich extension of the antisense ODN. Surprisingly, combination of the pPEG-ODN conjugate with the cationic agent, PEI, caused the antisense effect to vanish. Subsequent experiments without added PEI restored antisense. This composition produced the highest antisense response in the MCF-7 cells but in subsequent work produced similar results to other conjugates.

It was discovered that PEI added after micelle formation could inhibit the activity of antisense ODN delivered with amphiphilic PEG micelles. Surprisingly and unexpectedly, it was also shown that when the PEI was covalently grafted to the amphiphilic PEG the antisense activity was not impeded and was restored. It is not known why free PEI inhibits antisense activity when added to the composition after micelle formation. However, these embodiments show that the integrity of the particle formed in the micelle compositions is critical to intracellular transport and/or function of the antisense oligonucleotide in the cell.

In other experiments, it was discovered that micelles composed of the formula PEG[LX]yQ formed clear dispersions and resisted condensation at high N/P ratio in the PEI condensation assay. Likewise, micelles of formula PEG[LX]yNQ also formed clear dispersions at high N/P ratio. It was discovered that even at N/P ratios as high as 40:1, in which free polynucleotides formed opaque solutions the polynucleotide was protected from condensation.

These results and others, suggest that the PEG shielded micelle form particles which are effective in promoting translocation of the polynucleotide. However, the invention may also gain advantage from incorporation of one or more membrane permeabilizing agents (MPA). A membrane permeabilizing agent is a molecule which facilitates escape from intracellular compartments. The invention may comprise one or more membrane permeabilizing agents, grafted to the multifunctional PEG backbone via the spacer arm. In the present invention, trioxsalen may act as a membrane permeabilizing agent, provided that it is exposed at the surface of the micelle. The most preferred are those in which the active MPA is conjugated to the pendant PEG, such that its active surface is exposed. Hydroxychloroquine (HQ) was conjugated to the pendant PEG micelle by reacting with a diepoxy and then reacting the epoxy HQ conjugate with amine functions on the pendant PEG. In the present invention, the addition of free hydroxychloroquine was shown to improve uptake of polynucleotide by some of the shielded micelle preparations in COS7 cells but was not as effective as conjugation. Conjugation of hydroxychloroquine to the multifunctional PEG backbone is shown to enhance transfection possibly by increasing lysosomal escape and intracellular trafficking of the micelles.

PEI condensation experiments designed to explore the nature of the particles formed by linear PEG conjugates and multifunctional PEG conjugates showed that multibranched PEG provided significantly greater shielding than linear PEG. The cationic backbone polymer, polyethylenimine 25K daltons (PEI25K) was pegalated with a linear PEG and showed high turbidity with polynucleotides, producing greater sedimentation and turbidity than multibranched PEG-PEI at equivalent N/P ratio. Furthermore, linear PEG PEI conjugate condensed the nucleotide at equivalent N/P ratio as PEI alone, indicating that the shielding effect of the linear PEG was negligible.

In the formation of the shielded micelle, the polynucleotide may be either covalently or non-covalently attached to the amphiphilic polymer depending on the polynucleotide target to be delivered. In some embodiments it may be preferred to have a covalent linkage between the amphiphilic polymer and the polynucleotide and in other embodiments of the invention it may be preferred to have a non-covalent linkage. For example, covalently coupling the polynucleotide to the pPEG would be advantageous in providing a linkage to polynucleotides which do not associate effectively by charge such as peptide nucleic acids (PNA's) or MEO-ODNs. Covalently binding the polynucleotide to the amphiphilic polymer was shown to significantly reduce the background toxicity relative to some of the non-cationic carriers tested. Drug loading is also likely to be higher for micelles in which the polynucleotide is conjugated. For example, approximately 10 to 15 ODN's can be loaded per mol of carrier with 10-15 functional groups and a backbone polymer weight of 20K Daltons. Covalent coupling would likely give the highest drug loading possible.

The polymer can be selected from high molecular weight amphiphilic polymers or lower molecular weight polymers depending on the desired radius of the micelle, the desired clearance rate from the circulation, the delivery profile, and other pharmacokinetic properties. For example, in a parenteral formulation, it may be desired to increase the radius of the micelle by varying the molecular weight, to improve passive uptake and retention in selected areas of the body, such as a tumor, that are highly vascularized and porous to large molecules.

Amphiphilic, multifunctional PEG polymers have significant advantages for use as backbone polymers in the present invention. These polymers are highly water soluble, substantially inert, non-cytotoxic, and have low immunogenicity. The safety and pharmacokinetics of the PEG's are generally well established, making the amphiphilic polymers suitable for parenteral, oral, transmucosal, or pulmonary delivery.

Since toxicity is the primary barrier to current gene carriers, the present invention overcomes a significant pharmaceutical barrier to gene therapy. It has been discovered that the shielded micelles deliver polynucleotides to a cell nucleus without the requirement of additional agents. The conjugates of amphiphilic PEG-ODN's have been found to be non-toxic at the highest concentrations tested.

Micelles composed of pendant PEG-Protamine conjugate (pPEG-Protamine) have significant potential for clinical use as an approved substance because they would have dual application as either a non-viral polynucleotide delivery system or as a treatment for heparin overdose. Protamine sulfate is currently administered intravenously in a normal saline injection. Current dosage limits in humans are 50 mg administered over a 10 minute period (RXlist.com). The multi-functional PEG conjugates can improve the circulation time of protamines for enhanced oligonucleotide delivery, due to reduced clearance from the kidneys and impart other favorable pharmacokinetic properties.

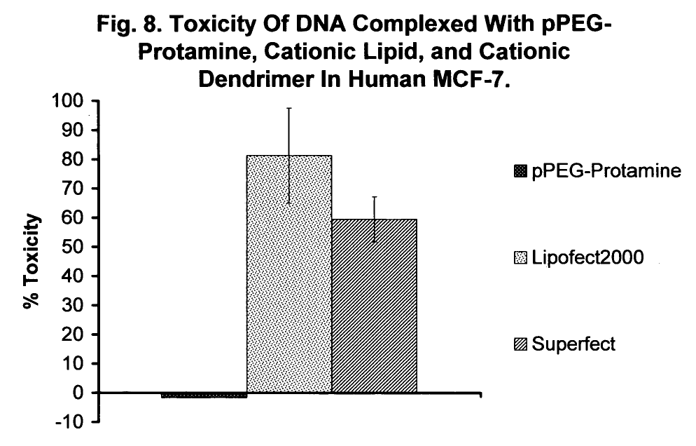

The multi-functional pPEG-Protamines herein are shown to have significantly greater transfection efficiency and lower toxicity than protamine alone. When formulated in a mixed micelle with PEG-hydroxychloroquine (PEG-HQ), the performance of the micelle system is further enhanced, about ten fold over protamine. In transfection experiments, the pPEG-Protamines are highly efficient carriers of large plasmids and have shown three fold higher luminescence than dendrimer (Superfect™ 15:1 EX021605). Furthermore, pPEG-Protamines are shown to have negligible toxicity while cationic lipid and dendrimer have 81% and 56% toxicity respectively (FIG. 8).

The pPEG-Protamine is a versatile transfection agent and is shown in-vitro to be effective at delivery of large plasmid DNAs as well as small RNA's. Plasmid DNA's have potential therapeutic value since these are showing promise for delivery of genes to cells but also for silencing genes by incorporating a sequence that produces an interfering RNA against the mRNA of a specific protein. Whereas siRNA's exhibit interference and silencing for short periods, the plasmid mediated shRNA (short hairpin RNA) approach has the advantage of stable, long-term silencing of the target gene. A plasmid encoding a therapeutic gene or interfering RNA can be delivered to a human patient using pPEG-Protamine micelle particles. The therapeutic plasmid can also include nuclear localization sequences such as SV-40, which are known to enhance targeting of the plasmid to the nucleus [Young, J. L., Benoit J. N., Dean D. A., Effect of a DNA nuclear targeting sequence on gene transfer and expression of plasmids in the intact vasculature, Gene Ther. 10:1465-1470 (2003)].

The present invention has application to pharmaceutical and biotech research, particularly in the in vitro or in vivo study of antisense oligonucleotides. The present invention would be particularly useful for applications that require negligible background, such that only the effect of the antisense oligonucleotide is observed. Hyperbranched-polyamidoamine pPEG (pPEG-PAM) is shown in the exemplary disclosure to have higher efficiency for transfection of plasmid DNA than commercially available cationic lipid or dendrimers. The pPEG-PAM is ideal for research applications in which high transfection efficiency and low background toxicity are needed.

The shielded micelle particles of the present invention can be formulated for parenteral, oral, transmucosal, or pulmonary delivery. The polynucleotide can be incorporated into the shielded micelle particles of the present invention and be administered intravenously in a physiological saline or other solution which is suitable for injection. In an oral formulation, the invention can be formulated in a tablet such as a pill or gel cap. For pulmonary administration, the invention can take the form of an aerosol or powder. The shielded micelle particles can also be administered via a hydrogel or other matrix deposited under the skin that can release the composition over time. Delivery of the composition through the skin can also be accomplished with a patch suitable for transdermal delivery. For ocular administration, the composition can be formulated in a suitable eye drop solution.

Examples of current polynucleotide therapeutics that can gain enhanced performance with the invention include antisense (ODN) and RNA interference (RNAi) therapy. Antisense oligonucleotides are currently in clinical trials for cancer treatment. The invention can be used to formulate a therapeutic oligonucleotide such as an antisense ODN or RNAi into shielded micelle particles for parenteral delivery. Incorporation of antisense oligonucleotides into shielded micelle particles of the invention is expected to enhance bioavailability, stability, and uptake.

Current phosphorothioated polynucleotide drugs show significant non-specific binding to blood proteins, leading to dose related side effects in clinical trials. Activation of the compliment system has been observed in cationic based systems such as PEI's and dendrimers [Plank C., Mechtler K., Szoka F., Wagner E., Activation of the complement system by synthetic DNA complexes: a potential barrier for intravenous gene delivery, Hum. Gene Ther. 7:1437-1446 (1996)]. In the design of the carrier it is important to reduce the potential for aggregation of erythrocytes or blood proteins to lessen the chance of embolism or of triggering the compliment system [Kircheis, R., Wagner E., Polycation/DNA complexes for in-vivo delivery, Gene Ther, Regul. 1(1) 95-114 (2000)]. The present invention can reduce non-specific binding of blood proteins to the oligonucleotides by PEG shielding of the polynucleotide within the micelle particle. The shielded micelles of the present invention can reduce non-specific binding to blood cells such as erythrocytes and significantly reduce the potential for activating the compliment system.

To improve the localization of shielded micelle particles in the body, the composition can be actively targeted by incorporating ligands on the micelle surface. Many targeting ligands are known in the art that are specific to cell surface receptors. These include antibodies, transferrin, folic acid, among others. Cell specific or tissue specific targeting of the shielded micelle can be enhanced by grafting a cell targeting moiety to the amphiphilic polymeric backbone of the present invention. If necessary, cellular targeting and or permeability can also be enhanced by synthetic viral peptide fragments. These peptide units are synthetic analogues of viral targeting peptides and are well known as potent cell targeting agents. Examples of cell targeting peptides include TAT, ANT, and RGD among others and are reviewed by Lochmann et al., Drug delivery of oligonucleotides by peptides, Eur. J. Pharm. 58:237-251 (2004).

The shielded micelles of the composition can improve the stability and bioavailability of antisense ODN's or RNAi's during parenteral administration. Other forms of administration which can be enhanced by the present invention include transdermal, oral, and pulmonary delivery. Oral delivery of oligonucleotides is presently hampered by poor uptake and rapid degradation in the small intestine. A current need exists for carriers in oral formulations, since carriers are shown to enhance stability in the intestine [Ferreiro M. G., Crooke R. M., Tillman L., Hardee G., Bodmeier R., Stability of polycationic complexes of an antisense oligonucleotide in rat small intestine homogenates, Eur. J. Pharm. 55: 19-26 (2003)]. Oral bioavailability can be enhanced with the present invention by formulating the polynucleotide in the shielded micelle particles, facilitating transport across cellular membranes and increasing polynucleotide drug half-life.

In clinical studies, ODN's are currently administered without a carrier. This is because there are currently no safe or effective carriers available for delivery of ODN's. To overcome poor uptake, bioavailability, and serum binding, ODN's are administered in doses that are much higher than required, leading to toxicity. Presently, serum binding is a significant problem, particularly with thioated ODN's and has required high dosing. Because non-specific binding to serum proteins and erythrocytes can potentially lead to side effects, a current need exists for the invention in reducing non-specific binding and improving localization of the polynucleotide in target tissues.

The invention can increase or enhance the performance of diester polynucleotides, diester ODN's or unthioated oligonucleotides which are presently non-viable for in-vivo use. Diester antisense oligonucleotides are in theory, a better therapeutic alternative than phosphorothioated ODN because they have higher specificity and lower non-specific toxicity. Diester ODN's also show less non-specific binding to serum proteins. Diester ODN's have stronger hybridization and melting points with complimentary targets such as mRNA targets, which may also improve effectiveness over thioated ODN's [Stein C. A., Subasinghe C., Shinozuka K., Cohen J. S., Physicochemical properties of phosphorothioated oligodeoxynucleotides. Nuc. Acids Res. 16:3209-3221 (1988)]. However, diester ODN's are currently not viable for in-vivo use because of rapid clearance and enzymatic susceptibility. However, the stability of diester ODN's in serum can be enhanced with the composition due to protection of the ODN within shielded micelle particles.

Ex-vivo gene therapy is currently an alternative approach to in-vivo and is a more advantageous treatment option in certain applications. In ex-vivo therapy, cells from a patient are transfected in-vitro and later implanted to elicit a specific gene expression in an organ, muscle, or joint. For example, ex-vivo gene therapy has been used successfully in humans to treat rheumatoid arthritis, hypercholesterolaemia, Alzheimer's, spinal cord injury, and Crohn's [Pap T. Gay R. E., Muller-L. U., Gay S., Ex vivo gene transfer in the years to come, Arthritis Res. 4:10-12 (2002), Bleshch, A., M. H Tuszynski, Ex vivo gene therapy for Alzheimer's disease and spinal cord injury. Clin. Neurosci 3:268-274 (1995), Amt gets Eur $1.8 million for ex vivo gene therapy for Crohn's Worldwide Biotech, Nov. (2003)]. Ex-vivo gene therapy is useful for selectively transducing cells that are difficult or impractical to transfect by in-vivo methods.

The present invention was used successfully in an ex-vivo gene therapy model to cause expression of an exogenous gene in mice. Green monkey kidney cells were transfected in tissue culture with multi-branched PEG-protamine nano-particles containing the firefly luciferase gene. The cells expressing the exogenous firefly luciferase gene were harvested and then injected into the mouse, intravenously, via the tail vein. Stable expression of the luciferase gene was detected in the mouse as bioluminescence in the lung, liver, and kidneys.

There are several fundamental criteria that relate specifically to the function of the successful polynucleotide delivery system. 1) The polynucleotide carrier must facilitate the transport of the polynucleotide across the cellular membrane. 2) The polynucleotide carrier must facilitate escape of the polynucleotide from intracellular compartments 3) The polynucleotide carrier must protect the polynucleotide from nuclease degradation, and finally, 4) The polynucleotide carrier should be non-cytotoxic by itself (low background) in cell culture at the concentration required for a minimum demonstrable effect of the polynucleotide.

The invention is a composition which facilitates uptake and delivery of polynucleotide drugs and therapeutic entities. The invention comprises a PEG shielded micelle particle in which the polynucleotide is incorporated by covalent or non-covalent means. The invention may further comprise targeting moieties which promote uptake into specific cell types and membrane permeabilizing agents which enhance endosomal escape. The invention provides a substantially safe, non-toxic, and non-immunogenic delivery system for polynucleotides.

### EXAMPLES OF THE BEST MODES FOR CARRYING OUT THE INVENTION

### Example 1

Synthesis of pPEGHdz914A (pendant PEG-Hydrazine). Dissolved 0.25 g pendant propionic acid PEG (Sun Bio, 20K, 15 mol propionic acids/mol PEG) 0.0125 mmol in 2 ml H2O. After 5 min. added 0.12 ml of 3.18 mmol/ml hydrazine pH7.5, giving a 2×mol ratio to PEG propionic acids. After 10 minutes, added 71.6 mg EDC (191 g/mol) or 0.37 mmol to give 2×mol ratio to PEG propionic acids. After 1 hour the reaction formed a gel at which point 0.12 ml of Hdz and 4 ml of H2O was added and the solution was sonicated 10 min 40-50 C. producing a clear solution and final molar ratio of 4×Hdz and 2×EDC per propionic acid. Final pH was 5.5. Allowed rxn to continue 14 hours @25 C before purification. Added 10 ml 100% isopropyl to give 19 ml total volume, then transferred to 3K Pall™ spin filtration membrane and centrifuged 60 min. at 4800 rpm. The solutions were concentrated to 3-5 ml in 1 hour then water was added to give 20 ml total volume, and centrifugation was repeated. After 3 repetitions, the solutions were transferred to glass vials and assayed for refractive index (RI) and total amine concentration.

### Example 2

Synthesis of pPEGHdz914B. Dissolved 0.25 g pPEG 0.0125 mmol into 5 ml H2O. After 5 min. added 20×mol ratio Hdz to propionic acids or 0.60 ml Hdz, 3.18 mmol/ml pH7.5. Let rxn stand for 10 min. then added 0.357 g 10×mol ratio EDC, 1.875 mmol, to produce a clear, pale yellow solution at pH 7.5. After 14 hours at 25 C, the reaction produced a partial polymerization, ˆb 10% of the total volume formed a gel. Purification was identical to the procedure used in pPEG HdzA.

### Example 3

Synthesis of pPEGHdz914C. Dissolved 0.25 g pPEG, 0.0125 mmol into 5 ml H2O. After 5 min, added 20×Hdz, 0.60 ml Hdz @3.18 mmol/ml, pH of Hdz 6.0. The pH of the solution was 4.0. After 10 min. added 10×EDC, 0.357 g, 1.87 mmol to give a final pH of 5.0. Reaction was continued for 14 hours prior to purification. Purification was identical to the procedure used in pPEG HdzA.

### Example 4

Synthesis of pPEGdiamine914D. Dissolved 0.125 g pPEG 0.00625 mmol in 1 ml H2O. After 5 min. added 1.0 ml 0.937 mmol/ml 1,6Hexane diamine pH5 to give 10×mol ratio to propionic acids and a final pH of 4.5. After 10 min. added 4.7 mol ratio of EDC, 0.438 mmol, 83.6 mg EDC. Continued rxn at 25 C for 14 hours prior to purification. Purification was identical to the procedure utilized in pPEG HdzA.

Table #1 shows assayed NH2/pPEG molar ratio for selected compounds.

TABLE 1

| Sample | Vol. | RI | NH2/pPEG |
|---|---|---|---|
| 914A | 2.95 | 32 | 12.6 |
| 914B | 5 | 24 | 1.2 |
| 914C | 4.2 | 32 | 0.9 |
| 914D | 1.25 | 50 | <<.5 |

### Example 5

Synthesis of pPEGHdz914A#2-Aldehyde-ODN

Combined 1.5 ml (79.5 mg, 0.00389 mmol) of the pPEGHdz914A with 10×mol of glutaraldehyde solution, 0.52 mmol, 0.208 ml of 25% (2.5 mmol/ml glutaraldehyde)+1 ml H2O. Reacted for 2.5 hours at 25 C and dialyze w/1K spectropore dialysis membrane for 4 hours in H2O. Aldehyde assay gave 1.04 mol aldehyde/mol pPEG.

Conjugation of 5′ amino labeled anti-sense oligodeoxynucleotide (ODNAS, G3139 Genta Inc.) and 5′amino labeled reverse sequence oligodeoxynucleotide (ODN-control) with pPEG914A#2aldehyde to give pPEG-AS1005 and pPEG-Control-1005.

In one reaction combined 0.250 ml of ODNAS 0.801 mg/ml with 0.5 mg of pPEG914A#2aldehyde. In a second reaction, combined 0.114 ml of ODNcontrol (reverse sequence) with 0.126 ml H2O and 0.5 mg of pPEG914A#2aldehyde. Both reactions were commenced at 53 C for 10 hours.

### Example 6

Antisense Experiment with pPEG-ODN Conjugate Micelles

MCF-7 human tumor cells were previously trypsinized from 80% confluent flask and dispersed into 96 well plates to give 6700 cells/well. After culturing 1 day, the pPEG-ODN-AS-1005 (G3139, Genta Inc.) or pPEG-ODN-control-1005 (reverse sequence) were diluted in 1×DMEM 10% FBS media and added to the cell culture plate in triplicate to give 80, 40 and 20 ug/ml final ODN in cell culture. After incubation of pPEG-ODN conjugates in culture, relative proliferation was measured by MTT assay and absorbance read at 570 nm. The pPEG-ODNAS and pPEG-ODN-control were found to inhibit MCF-7 proliferation by 12.5% and 6% respectively. At 200 ug/ml the same conjugates were found to inhibit proliferation by 32% and 12.5% respectively. pPEG914A#2aldehyde showed no toxicity at between 200 and 800 ug/ml in cell culture.

### Example 7

Synthesis of pPEG-NHS (pendant PEG N-hydroxy succinimide ester). Dissolve 0.25 g pPEG (0.0125 mmol) in 1 ml H2O to produce a clear solution. Add 15×mol ratio of EDC, 71.6 mg and after 10 min. formed gel. Added approx. 2 ml H2O to the reaction caused the polymeric gel to dissolve. After 20 min. 120 mg of N-Hydroxy succinimide (115 mg/mmol) or 5.3 mol ratio was added, and the rxn was allowed to stand @25 C for 2 hours. Formation of pPEG-ethylcarbazate/Assay of pPEG-NHS activity. The relative activity of the N-Hydroxy Succinimide activated ester was assayed using ethyl carbazate as a standard. The pPEG-NHS product was diluted serially in H2O at 0.25×, 0.125× etc to 0.1 ml final volume. Added 0.05 ml 1 mM ethyl carbazate to all wells and allowed reaction for 30 min at 25 C. followed by addition 0.1 ml 0.1M Na3PO4 and 0.05 ml 0.125% TNBS (picryl sulfonic acid). Let stand 10 min then read A515. Approx. first 4 wells showed the absence of primary amine groups, indicating that ethyl carbazate was consumed in rxn with pPEG-NHS. Purification of pPEG-NHS by G25 Load entire product volume onto column (2 ml) and pre-elute 3 ml. Collect 25 drops/well/0.75 ml, eluent=H2O. Absorbance at 230 nm and 290 nm produced single broad peak.

Antisense experiment with shielded, conjugate micelles in MCF-7 proliferation assay. MCF-7 human tumor cells were trypsinized and seeded into 96 well plates to give 6150 cells/well. ODN conjugates were prepared by combining 50 ul of 0.801 mg/ml ODN AS 5′amine (G3139, Genta Inc.) with 8 ul of 10 mg/ml pPEGNHS 1104B. The product was dried completely at 48 C for 2 hours in styrene wells. Combine 27 ul of 1.47 mg/ml ODN control 5″amine with 8 ul of 10 mg/ml pPEGNHS 1104B. Products were dried completely at 48 C for 2 hours and reconstituted in 0.2 ml H2O. Diluted each pPEGNHS-ODNAS (G3139, Genta Inc.) conjugate or pPEGNHS-ODN control (reverse sequence) in media and dispensed into triplicate wells containing tumor cells. Cell proliferation was quantified by MTT viability assay at A570. Average OD570 from triplicate wells was 0.409 and 0.421 for pPEGNHS-AS and pPEGNHS-control respectively.

### Example 6

Synthesis of pPEG-ethanol amine. Fifty microliters of each fraction was then combined with 0.05 ml 1 mM ethanol amine and incubated 30 min. followed by addition of 0.1 ml Na3PO4 and 0.05 ml 0.125% TNBS. The reaction produced surprising results. Instead of the expected reduction in amine signal as shown with ethyl carbazate, ethanol amine produced a spike in TNBS signal where pPEG-NHS concentration was highest. Furthermore, ethanol amine did not react with TNBS in other wells that did not contain the pPEG-NHS.

### Example 7

Synthesis of pPEG-polyethyleneimine p1020H-Combined 50 mg pPEG with 2 ml H2O. Added 1.0 ml of 0.111 mmol/ml PEI ˆ2K branched, neutralized to pH7 in H2O waited 10 min then added 0.07 ml EDC 1.047 mmol/ml. Allowed rxn 24 hours, dialyzed in 6 inch 10K spectropore tubing against 1000 ml water for 48 hours, replacing H2O every 24 hours. Repeated dialysis in 25K tubing for 3 hours.

### Example 8

Antisense experiment with pPEG-PEI1020H shielded micelles against MCF-7 tumor cells. MCF-7 human tumor cells were trypsinized and seeded into 96 well plates to give 6150 cells/well. pPEG-PEI1020H was combined at 0.021 pPEG/phosphate molar ratio with either ODNAS (G3139, Genta Inc.) or ODNControl (reverse) sequence in the following procedure. Combine 43 ul of 0.81 mg/ml ODNAS+7.7 ul pPEG1020H @5.8 mg/ml+50 ul H2O, then add 100 ul H2O. Combined 23 ul of 1.47 mg/ml ODNcontrol+7.7 ul pPEG1020H @5.8 mg/ml+70 ul H2O then added 100 ul H2O and subsequently diluted in media and dispensed into triplicate wells containing tumor cells. After an incubation period, the relative proliferation was assayed using MTT, and absorbance read at 570 nm. The pPEG-PEI1020H combined with the ODN AS or ODN control was found to inhibit MCF-7 proliferation by approximately 50% or 33% respectively at 80 ug/ml final in cell culture.

### Example 9

Demonstration of PEG shielding of polynucleotides in pPEG-micelles (EXP924). In this assay the N/P ratio was fixed at 44:1 for all samples. Prepared dilution series of four different pPEGHdz stocks at 0.5× 0.25× 0.125× etc in H2O to 25 ul final. Added 70 ul of 1 mg/ml HSDNA (herring sperm DNA pH 7.0). Added 40 ul of 10 mg/ml PEI 25K (prepared in 0.1M KP pH 6.5). PEI25K stock was previously prepared as 10 mg/ml (unneutralized) in 0.1M KP pH 6.5. Solutions were prepared in styrene wells. Turbidity was read at A410. Another pPEGHdz, pPEGHdz823 was prepared, but was ineffective at forming shielded micelles. pPEGHdz914A, C were found to be highly effective shielding and about 10 times more effective than pPEGHdz914B.

Table #2 shows OD410 of selected pPEG-Hdz compounds with polynucleotides in the presence of added PEI at 44:1 N/P ratio.

TABLE 2

| Stock | A410 | | | | | | |
|---|---|---|---|---|---|---|---|
| pPEGHdz823 | 1.105 | 1.042 | 1.002 | 0.981 | 0.974 | 0.983 | 0.968 |
| pPEGHdz914A | 0.08 | 0.089 | 0.118 | 0.186 | 0.318 | 0.531 | 0.936 |
| pPEGHdz914B | 0.331 | 0.44 | 0.669 | 0.911 | 1.04 | 1.089 | 0.931 |
| pPEGHdz914C | 0.068 | 0.077 | 0.1 | 0.157 | 0.262 | 0.443 | 0.985 |

### Example 10

Synthesis of pPEG 1,6 hexane diamine compounds. p1020E-Combine 50 mg pPEG with 2 ml H2O. Add 0.40 ml of 0.937 mmol/ml 1,6hexane diamine pH7 in H2O wait 10 min then add 0.07 ml EDC 1.047 mmol/ml. Allow rxn 24 hours, dialyze in 6 inch 2K spectropore tubing against 1000 ml water for 48 hours, replacing H2O every 24 hours. p1020F-Combine 50 mg pPEG with 2 ml H2O. Add 0.07 ml EDC 1.047 mmol/ml wait 10 min, then add 0.40 ml of 0.937 mmol/ml 1,6hexane diamine pH7 in H2O Allow rxn 24 hours, dialyze in 6 inch 2K spectropore tubing against 1000 ml water for 48 hours, replacing H2O every 24 hours. p1020G-Combine 50 mg pPEG with 2 ml H2O. Add 0.40 ml of 0.937 mmol/ml 1,6hexane diamine pH6.5 in H2O wait 10 min then add 0.07 ml EDC 1.047 mmol/ml. Allow rxn 24 hours, dialyze in 6 inch 2K spectropore tubing against 1000 ml water for 48 hours, replacing H2O every 24 hours.

### Example 11

Demonstration of PEG Shielding of Polynucleotides in pPEG-Micelles (EXP1020).

Samples were diluted directly from stocks to give 1×, 0.5×, 0.25× etc in 50 ul final vol. Added 70 ul of 1 mg/ml HSDNA 428A pH7, followed by 35 ul of 1 mg/ml PEI25K pH6.68. Turbidity was read after 15 min @OD410 nm. Surprisingly, shielded micelles of 1020G were roughly 10 times more effective than 1020E or 1020F (table 3).

Table #3 shows OD410 of selected pPEG1,6HD in presence of PEI at 4:1 N/P ratio.

TABLE 3

| Sample | pPEG/P Mol ratio | OD410 |
|---|---|---|
| pPEG1,6HD 1020E | 0.228 | 0.0161 |
| pPEG1,6HD 1020F | 0.222 | 0.0161 |
| pPEG1,6HD 1020G | 0.0169 | 0.197 |
| pPEGPEI1020H | 0.0012 | 0.244 |

### Example 12

Experiment comparing shielding capacity of particles formed with pendant PEG backbone and cationic polymer backbone by turbidity at A410 (EX121604). Compounds pPEGPEI's 1123A, 1123B were synthesized by similar method shown previously for pPEG1020H. pPEGHdz1208A was synthesized as in pPEG914A. pPEG-DMS was prepared by reacting pPEGHdz914A with dimethylsuberimidate (DMS) at pH12 in H2O for 16 hours and purified by dialysis in 2K membrane. PEG-PEI1209B was prepared by reacting a 25K PEI backbone polymer with a linear, diepoxy 23K PEG at 25 C for 16 hours. Purified on 1×12.5 cm S500 column, eluent 30% MEOH 5 mMHCl.

Table #4 shows eight synthesized multi-branched PEG amino compounds.

TABLE 4

| | | mg/ml | |
|---|---|---|---|
| Stock | Description | [pPEG] | Notes |
| pPEGHdz914B | pPEGHdz | 40 | |
| pPEGHdz914C | pPEGHdz | 53 | |
| pPEGPEI1020H | pPEGPEI2K | 5.8 | likely <<5.8 |
| pPEGPEI1123A | pPEGPEI1.2K | 15 | < or = to 15 |
| pPEGPEI1123B | pPEGPEI.8K | 15 | < or = to 15 |
| pPEGHdz1208A | pPEGHdz | 14.9 | |
| pPEGDMS1208D | pPEGDMS | 8.3 | |
| PEIPEG1209B | PEI-PEG | 110 | |

Prepared dilution curve of stock 1×, 0.5×, 0.25×, 0.125×, etc to 50 ul final in H2O. Added 70 ul 1 mg/ml HSDNA (prev. heated 80 C), then added 36 ul 1 mg/ml PEI 25K. Read A410.

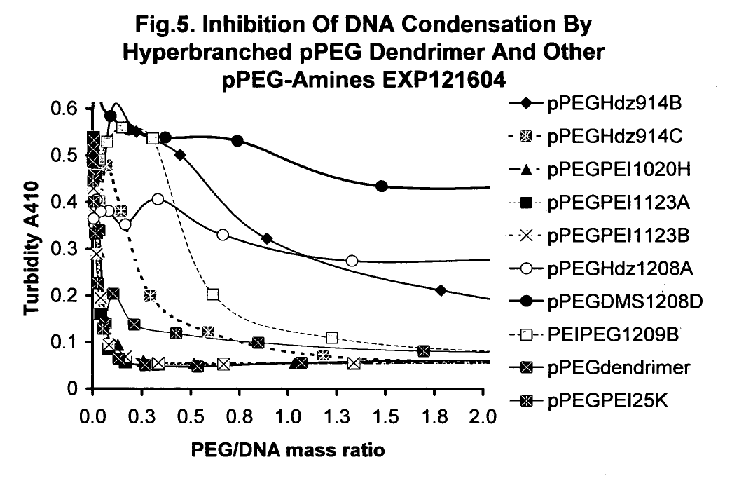

Results were plotted as PEG/DNA mass ratio vs A410 (see FIG. 5). The pPEGPEI's show superior binding affinity and shielding toward DNA, 20 fold better than pPEGHdz914C and 50 fold better than PEIPEG 1209B. PEI-PEG1209B never produced equivalent shielding to pPEGHdz or pPEGPEI's since turbidity was observed in all wells. pPEGPEI's showed maximum shielding at up to 0.002 pPEG/phosphate mol ratio. pPEGHdz914C showed maximum shielding at 0.04 pPEG/phosphate mol ratio.

### Example 13

Cytotoxicity Study of Shielded Micelles (EX1104).

Cells were trypsinized and seeded into 96 well plates to give 6150 cells/well. Stock solution of pPEGNHS or pPEGHdz914C were diluted 2× in 2× media to give 1×DMEM 2.5% FBS, directly transferred to cell culture and incubated for 72 hours, and assayed by MTT to determine cell viability. MTT values were averaged for triplicate wells. Final [pPEGNHS] and [pPEGHdz914C] ranged from 0.67, 0.33 and 0.15 mg/ml and gave MTT A570 (0.74, 0.74, 0.74) and A570 (0.78, 0.77, 0.73) respectively. No toxicity was observed.

### Example 14

Synthesis of pPEG-Aldehyde-trioxsalen amine (TX). (EXP616). Combined 0.00144 mmol (0.6 ml 48 mg/ml) pPEG Hdz (Nat29) 0.0072 mmol amine with a 10× ratio or 0.072 mmol of glutaraldehyde 0.029 ml of 25% glutaraldehyde. Allowed reaction for 2 hours, then purified on G50 column in 96 well plate. Purification on G50 of pPEG-Aldehyde yielded two peaks, one for the pPEG-Aldehyde and one of pure glutaraldehyde. pPEG-Aldehyde was assayed with an amine standard, ethyl carbazate. Combined 10 micro of sample with 10 micro of 1 mM ET-Carbazate, reacted for 1 hour, then added 20 micro of 1M Na3PO4 pH 14 followed by 10 micro of 0.125% TNBS and read A515. The aldehyde peak was clearly visible by the assay. The assay also showed reactivity of aldehyde groups on the pPEG, demonstrating successful derivatization. The fractions were pooled from collection wells B1-B12 and C1-C3 to give 3.9 ml total in H2O and 0.0072 mmol nominal aldehyde (assume each amine reacted). Added 0.648 ml 70:30 DMSO/DMF Trioxsalen amine (2.3 mg, 0.0078 mmol mw 293 g/mol). The reaction was heated 55 C 3 hours then concentrated at 25 C 12 hours to 0.4 ml 100% DMSO. Prior to addition to the column the concentrate was diluted with 0.6 ml H2O. Purify on G50 1×13 cm 10 drops/well pre collect 1 ml. G50 purification of pPEG-Hydrazone-Aldehyde-TX showed two distinct peaks corresponding to the TX-functionalized pPEG and unreacted TX. As expected the conjugated TX showed higher fluorescence at 485 excitation wavelength than at 340 nm. The unconjugated trioxsalen was observed in the void volume peak and exhibited unshifted fluorescence peak at 340/580 nm. Note: The shift in fluorescence of TX due to conjugation was previously observed in the reaction with glutaraldehyde (Aid). pPEG-Hyd-Ald-TX was Pooled A12, B1-B12 and C1-C3 to give 4.48 ml and 0.0072 mmol (nominal) of the TX.

### Example 15

Synthesis of pPEG-Ald-TX-ODN Conjugate

Combined 0.35 ml or 0.0583 micromol of each thioated AS (G3139, Genta Inc.) or control ODN with 0.022 ml pPEGTX616 (0.035 micromol trioxsalen amine, TX) and uv crosslinked with 365 nm light for 2 hours in a shallow pan. Each solution was then quantitatively transferred to S75 column 1×13 cm e=H2O and purified of free pPEG-TX. After S75 purification the pPEG-ODN's were irradiated for 1 hour at 365 nm to sterilize and transferred to glass scintillation vial. The products were then heated 0.5 hour at 60 C. The concentration of ODN in pPEG conjugates was assayed by absorbance and calculated with thioate ODN standard curve to give 0.103 mg/ml and 0.114 mg/ml for pPEG-ODN AS and pPEG-ODN control respectively.

### Example 16

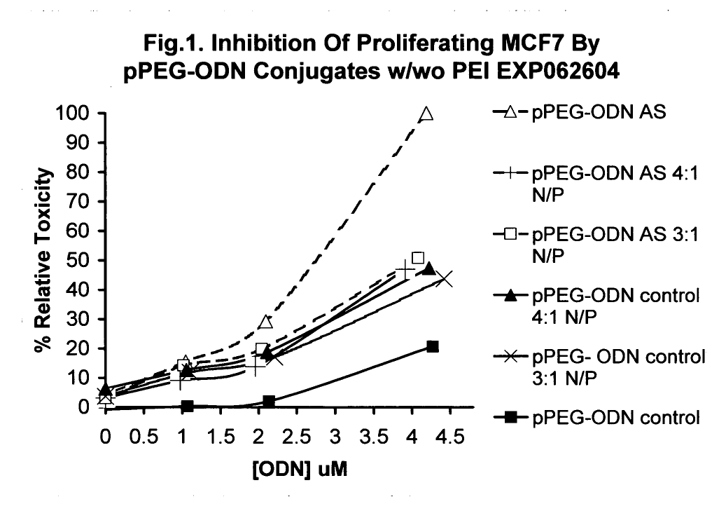

Antisense Activity of pPEG-Ald-TX-ODN Shielded Micelles (with and without Added PEI) EXP062604 (FIG. 1).

MCF-7 human tumor cells were previously trypsinized from 80% confluent flask and seeded into 96 well plates to give approx. 10000 cells/well. pPEG-ODN conjugate micelles were prepared by combining 0.146 ml and 0.132 ml of pPEG ODN “anti-sense” G3139 Genta Inc. (pPEGODNAS) or pPEGODNcontrol (mismatch) respectively with either water or PEI to give 4:1, 3:1, and 2.5:1 or 0.0 N/P ratio in 165 ul H2O. After culturing cells 1 day, the pPEG-ODNAS and pPEG-control conjugate micelles were diluted in 1×DMEM 10% FBS media and added to the cell culture plate in triplicate to give 45, 22 and 12 ug/ml final ODN in 50 ul. After three days incubation, cells were incubated in fresh 1× media 1 hour, aspirated, and replaced with 1× media containing 0.5 mg/ml MTT for 2 hours. Absorbance was read at 570. PEG-AS conjugate micelles were found to inhibit proliferation by 100% at 45 mg/ml final ODN. pPEG-control showed 20% inhibition. pPEG-ODNAS and pPEGcontrol micelles with added PEI showed inhibition of antisense activity and showed higher non-sequence specific toxicity.

### Example 17

Small Molecule Shielding of pPEG Micelles (EX 1104)

The pPEG polynucleotide micelle was prepared at final 0.4 mg/ml HSGDNA (herring sperm genomic DNA, Promega) and 4.3 mg/ml pPEGHdz914C in water to give 0.177 pPEG/phosphate mol ratio and N/P ratio of 5.3. To a 0.5 ml aliquot of pPEGHdzHSGDNA micelles, added 10 ul 207 uM doxorubicin HCl in water. For control, 10 ul of 207 uM doxorubicin HCl was combined with 0.5 ml of 0.4 mg/ml HSGDNA. Cells were tripsanized and seeded into 96 well plates to give 6150 cells/well. Solutions were diluted 2× in 2× media to give 1×DMEM 2.5% FBS, directly transferred to cell culture and incubated for 72 hours, and assayed by MTT to determine cell viability. pPEGHdzHSGDNA showed no toxicity MTT at OD570(0.6). HSGDNA-DOX showed moderate toxicity OD570(0.35) and pPEGHdzHSGDNA-DOX showed the highest toxicity, killing 100% cells OD570(0.08). It was found that addition of doxorubicin after formation of the micelle caused the highest toxicity due to the shielding of the polynucleotide from intercalation by doxorubicin.

### Example 18

Anti-Proliferation of MCF-7 Tumor Cells by siRNA-pPEG Micelles

Combined 0.00113 mmol (0.47 ml 48 mg/ml) pPEGHdz (Nat29) 0.0056 mmol amine with a 10× ratio or 0.056 mmol of glutaraldehyde 0.022 ml of 25% glutaraldehyde. Allow reaction for 2.5 hours, then purify on G50 column. Pre-collect 0.47 ml and collected 10 drops/well/0.28 ml. Measured OD @230 290 nm then assayed for glutaraldehyde and pooled the peak to give 4.7 ml total yield. Aldehyde assay: combine 10 micro sample with 10 micro 1 mM ethyl carbazate, incubate 1 hour 25 C then add 20 micro 0.1M Na3PO4 pH 14 followed by 0.01 ml 0.125% TNBS. Read after 10 minutes A515.

Prepared siRNA(sense)-pPEG721A by combining 0.3 mg siRNA (sense 5′amine labeled, mw 9900) at 0.86 mol aldehyde:amine ratio with 22 ul pPEGAld for 3 hours at 70 C then concentrated at 25 C. Prepared siRNA(sense)-pPEG721B by combining 0.25 mg siRNA(sense) 5′amine with 36 ul pPEGAld at 1.68 mol aldehyde:amine ratio for 3 hours at 55 C. RNA antisense was combined with siRNA-pPEG721A and B products, and melted at 70-80 C to form a double stranded hybrid of the siRNA antisense and the conjugated siRNA sense strand. Both siRNA (sense) and siRNA antisense hybrid of siRNA-pPEG721B showed high activity (50-60% inhibition) against MCF-7 proliferation cell culture reduction while the identical siRNA-pPEG721A synthesis, prepared at 70 C showed little activity (20%, equivalent to H2O control) at equivalent [siRNA] and [pPEG] carrier in cells, suggesting that higher temp synthesis inactivated the siRNA.

### Example 19

Synthesis and Characterization of Hyperbranched Polyamido pPEG (pPEG-PAM).

Hyperbranched polyamido PEG (pPEG-PAM, MY605A) was synthesized with approximately 10-15 pendant, polyamido amine dendrimer groups. In this case, multi-branched PEG (pPEG propionic acid 20K from Sun Bio Inc.) was used as the core polymer. About 100 mg of polyamidoamine dendrimer g=5 (Dendritech Inc.) was reacted with 30 mg of pPEG using EDC as a catalyst. The product was purified by dialysis and was compared for transfection efficiency against the dendrimer, lipofectamine™2000 (Invitrogen Inc), and SuperFect™ transfection reagent (Qiagen Inc.). Hyper-branched polyamidoamine PEG (pPEG-PAM, cpd MY605A) was prepared in water at 10:1, 15:1, and 20:1 mass ratio to plasmid DNA, Superfect™ and lipofectamine™2000 were prepared at the optimal 15:1 and 5:1 mass ratio respectively, and the dendrimer was prepared at optimal 10:1 mass ratio. Each solution was then diluted in 2× media and added to 90% confluent COS7 cells, to give 0.04 ug PGL3 plasmid/well, incubated for 48 hours under standard 37 C, 5% CO2 and then assayed for luminescence (20 ul reporter lysis buffer, followed by 40 ul of luciferase assay reagent, Promega Corp.). The resulting luminescence was measured on Synergy HT1 spectrophotometer (BioTek Inc), and results (avg of duplicates) are shown in the table below. In other assays (data not shown), the hyper-branched polyamidoamine PEG at 0.06 mg/ml (equiv. dendrimer mass) was shown to have three fold higher luminescence than the 0.09 mg/ml dendrimer (EXJ0505).

Table #5 shows a comparison of transfection efficiency of hyperbranched pPEG-dendrimer (pPEG-PAM) with monomer and commercially available transfection agents using luminescence.

TABLE 5

| Sample | Formula. | Lumens |
|---|---|---|
| MY605A_10 | pPEG-PAM | 10706 |
| MY605A_15 | pPEG-PAM | 13402 |
| MY605A_20 | pPEG-PAM | 17176 |
| Superfect ™ | dendrimer | 9673 |
| Lipo2000 | Cat. lipid | 3777 |
| dendrimer | Monomer G = 5 | 10032 |

The hyper-branched polyamido pPEG was assayed against dendrimer in the PEI condensation assay to demonstrate micelle stability and PEG shielding (FIG. 5). Samples were diluted in 50 ul water followed by addition of 70 ul 1 mg/ml HSDNA and 36 ul 1 mg/ml PEI25K to give N/P ratio of PEI/DNA of 4.2. Turbidity was measured with Synergy HT1 (BioTek) at A410 in uv-clear falcon™ plate. Results are shown below (table 6).

Table #6 shows difference in structure and binding of pPEG-PAM (MY605A) compared with PAM monomer (dendrimer), and pPEG-PEI's in PEI condensation assay.

TABLE 6

| Sample | PEG/DNA | polyamido/DNA | OD410 |
|---|---|---|---|
| MY605A | 0.27 | 0.89 | 0.051 |
| pPEGPEI25K | 0.21 | | 0.138 |
| pPEGPEI1020H | 0.26 | | 0.068 |
| dendrimer | | 0.93 | 0.5090 |

The hyper-branched polyamido pPEG produced equivalent PEG shielding to pPEG1020H indicating that it forms a very stable micelle with DNA, whereas the dendrimer formed a very different particle with DNA, a polyplex, which aggregated to form a highly turbid solution. Turbidity was approximately 10 fold greater (0.509) for the dendrimer compared with the hyper-branched polyamido pPEG (0.051) at comparable final polyamido amine mass to DNA ratio, which is confirms that the polyamido units are conjugated to the pPEG branches.

### Example 20

Synthesis of pPEG-Protamine (pPEG11105C)

Dissolved 100 mg of protamine III (Herring, Sigma-Aldrich) in 2 ml H2O followed with 50 mg pPEG propionic acid. After 5 minutes added 25 mg EDC. Allowed rxn for 16 hours 45 C, solution was transparent at 40 C, highly turbid at 25 C. Added 8 ml H2O at 25 C, formed a stable, transparent solution at 25 C (approx. 10 mg/ml protamine III). Dialyzed 16 hours in 25K Spectropore™ membrane against H2O.

### Example 21

Synthesis of Hydroxychloroquine-Epoxy Conjugate (HQ-Epoxy)

HQ-epoxy 11205A. Combined 0.433 g (1 mmol) of hydroxychloroquine (Aldrich) with 4 ml H2O containing 0.5M NaOH. Formed an opaque, insoluble slurry of HQ, due to high pH. Added 1 mmol of diepoxyoctane (0.145 ml of 97% solution, Aldrich) and stirred for 10 min at 70 C to form a transparent, yellow precipitate.

HQ-epoxy 11205B. Dissolved 0.5 mmol HQ, 0.216 g with 1 ml H2O and 0.4 mmol DEO, 0.056 ml 97% DEO (mw 142) Add 250 ul of 1M NaOH, to give approx. 0.25M NaOH and heated overnight at 45 C to yield a slight yellow transparent solution.

HQ-epoxy 11205C. Dissolved 0.5 mmol HQ, 0.216 g in 1 ml H2O and 0.1 mmol DEO, 0.0142 ml and heated 16 hours 40 C to give a transparent, colorless solution.

### Example 22

Synthesis of pPEG-Hydroxychloroquine (pPEGPEIHQ-11405A)

Combine 0.01 ml of 300 mM HQepoxy(11205A) with 0.6 ml of pPEG1123A (15 mg/ml pPEG, 9.8 mg/ml PEI) and heat at 45 C 16 hours in 20 ml glass vial. Dialyze product in 3 inch, 2K spectropore™ tube against water for 16 hours. pPEGHQ-11405B Combined 0.01 ml of 300 mM HQepoxy (11205A) with 0.8 ml of pPEGHdz1208A (mg/ml pPEGHdz) and heated at 45 C 16 hours in 20 ml glass vial. Dialyzed product in 3 inch, 2K spectropore™ tube against water for 16 hours.

pPEGPEIHQ-11405C

Combine 0.01 ml of 300 mM HQepoxyl1205C with 0.6 ml of pPEG1123A (15 mg/ml pPEG, 9.8 mg/ml PEI) and heat at 45 C 16 hours in 20 ml glass vial. Dialyze product in 3 inch, 2K spectropore™ tube against water for 16 hours.

Assay of [HQ]. Prepare standard curve of 10 mM hydroxychloroquine in water at 1×, 0.5×, 0.25× etc in 0.2 ml final volume. Products showed characteristic double absorbance peak for HQ at 328 and 342 nm or were slightly shifted due to conjugation. Read OD @328 nm and 342 nm. Prepare sample in 0.2 ml, read A342 nm. Calculate sample concentration with linear regression curve. Values are shown below in table.

Table #7 shows selected multi-branched pPEG-hydroxychloroquine conjugates and assayed HQ, pPEG, and PEI.

TABLE 7

| | | QS | | | mg/ml | mg/ml |
|---|---|---|---|---|---|---|
| sample | description | vol. | RI | mM[HQ] | [pPEG] | [PEI] |
| 11405A | pPEGPEIHQ | 1 | 14 | 0.2534 | 6.18 | 4.0353 |
| 11405B | pPEGHdzHQ | 1 | 10 | 0.5188 | 8.30 | N/A |
| 11405C | pPEGPEIHQ | 1 | 14 | 0.0431 | 6.18 | 4.0353 |

### Example 23

Comparing Transfection Efficiency of Protamine, pPEG-Protamine, pPEG-PEI, and pPEG-Protamine Mixed Micelles (EX012805).

COS-7 cells were trypsinized and seeded into 96 well plates to give approx. 13K cells per well. Prepare samples in 1× media with carrier and pGL3 plasmid DNA (Promega) w/wo free HQ or PEG-HQ (synthesized previously). After cells were 90% confluent, replaced with 50 ul fresh media and add 50 ul of each sample containing DNA complexes to give 100 ul total and 0.56 ug DNA/well. Incubated 48 hours, then assayed for luminescence. Aspirate media, and carefully rinse cells with 50 ul of HBSS Ca2+ free. Add 20 ul of 1× Promega lysis buffer, incubate 5 minutes 25 C and freeze 20 min at −85 C repeat freeze/thaw, then add 80 ul of luciferase assay reagent (Promega) and read luminescence at S=150. Average luminescence was taken from triplicate values. Mixed micelle pPEG-11105C_{—}80 (mass ratio carrier:DNA=80:1) with 50 mM PEG-hydroxychloroquine, showed about 10 fold higher luminescence than protamine alone. Surprisingly, addition of free HQ caused significant reduction in performance of pPEG-Protamine (pPEG11105C).

Table #8 shows the affect of free HQ and PEG-HQ on transfection efficiency of free protamine, pPEG-Protamine conjugate (pPEG11105C), and pPEG-PEI2K (pPEG1020H).

TABLE 8

| Sample | LUM | [HQ]@50 mM |
|---|---|---|
| Protamine_15 | 2067 | none |
| Protamine_67 | 2120 | none |
| Protamine_15 | 2095 | Free HQ |
| Protamine_67 | 3939 | Free HQ |
| pPEG11105C_16 | 3884 | Free HQ |
| pPEG11105C_80 | 6487 | Free HQ |
| pPEG11105C_16 | 6560 | none |
| pPEG11105C_80 | 12278 | none |
| pPEG11105C_16 | 5936 | PEG-HQ |
| pPEG11105C_80 | 21593 | PEG-HQ |
| pPEG1020H | 11309 | PEG-HQ |

Prepared 12.5 ug (0.175 ml 0.0716 mg/mlPGL3) of PGL3 (Promega Inc.) with 0.1 ml H2O and 62.5 ul of Superfect™ (3 mg/ml), diluted in 5 ml 1× media (10% FBS), aspirate wells containing 90% confluent COS-7, dispensed 50 ul plasmid per well to give 0.10 ugPGL3/well. Prepare anti-PGL3 siRNA (Dharmacon Inc.) with carriers as indicated in water at optimal mass, N/P, or PEG/phosphate ratios, w/wo PEG-HQ as indicated, then diluted with 2× media to give 1×. The RNA:Plasmid mol ratio was 174:1. Fifty microliters of sample was added immediately after adding PGL-3 complexes to give 100 ul 1× media, 50 mM PEG-HQ, and 50 nM siRNA final per well. COS-7 cells were incubated for 48 hours and luminescence assayed. For luminescence, aspirated media, rinsed cells with 100 ul Ca+free HBSS and added 20 ul 1× reporter lysis buffer RLB (Promega Inc.). Incubated 25 C 5 min, freeze −70 10 min., thaw 25 C 10 min, freeze −70 C 10 min, thaw 25 C 10 min. Add 80 ul luciferase assay reagent (Promega Inc.) read plate on Synergy HT1 microplate reader (BioTek) @ S=150.

### Example 25

Comparing Function of Linear PEG-Protamine and Multi-Branched pPEG-Protamine (EX030705).

Two linear PEG-Protamines were prepared by reacting a bifunctional epoxy PEG (20K) with either Protamine 25K or Protamine 2K previously fractionated and purified by dialysis. Multi-branched pPEG-Protamine (pPEG11105C), linear PEG-Protamine 25K, or linear PEG-Protamine 2K was prepared as mixed-micelle particles in HBSS with double stranded siRNA, diluted in DMEM media, and incubated in confluent COS7 cells at 50 nM RNA final and 0.288 nM of PGL3-dendrimer (15:1 mass ratio) particles. After 48 hours incubation the luciferase assay was performed. Aspirated media, rinsed cells with 100 ul Ca+ free HBSS and added 20 ul 1× reporter lysis buffer RLB (Promega). Incubated 25 C 5 min, freeze −70 10 min. repeat. Add 80 ul luciferase assay reagent (Promega) read plate @ S=150 Luminescence of samples was measured with Synergy HT1 plate reader (BioTek). At comparable protamine: DNA mass ratios, linear PEG-Protamine samples showed 100% toxicity to cells with low levels of luminescence, while pPEG11105C micelles showed negligible toxicity and significantly higher luminescence.

Table #9 shows a comparison of toxicity to COS7 cells of linear PEG-Protamines and pPEG-Protamines at equivalent mass ratio.

toxicity to cells with low levels of luminescence, while pPEG 1105C micelles showed negligible toxicity and significantly higher luminescence.

Table #9 shows a comparison of toxicity to COS7 cells of linear PEG-Protamines and pPEG-Protamines at equivalent mass ratio.

TABLE 9

| Sample | LUM | [PEGHQ] | mass ratio | toxicity |
|---|---|---|---|---|
| pPEG11105C_100_C | 60546 | 0.054 | 100 | neg. |
| pPEG11105C_100_C | 59400 | 0.163 | 100 | neg. |
| pPEG11105C_125_C | 61209 | 0.054 | 125 | neg. |
| PEGProt25K | 14684 | | 98 | 100% |
| PEGProt2K | 12941 | | 110 | 100% |
| PEGProt25K | 11603 | | 101 | 100% |
| PEGProt2K | 11562 | | 113 | 100% |

### Example 26

In-Vivo Gene Expression with pPEG-Protamine:Plasmid Nanoparticles Encoding the Firefly Luciferase Gene (EX110705).

Plasmid DNA's encoding the firefly luciferase gene were complexed with pPEG-protamine to form pPEG-Protamine:plasmid nano-particles. The pPEG-protamine: plasmid nano-particles were then administered to mice intravenously via tail vein injection. Luciferase activity due to expression of the gene was subsequently monitored in lung, liver, spleen, and kidney. As positive control, other commercial available carriers were also prepared with the identical plasmid and administered to mice.

Materials used: pPEGprotamine92305 40 mg/ml (prepared identically to 11105C), plasmid DNA 1 mg/ml (pGWz₁₃ luc plasmid encoding luciferase gene with cmv promoter, Aldevron Inc.), sterile H2O, sterile saline solution 20%. 80 ul of 0.05% PAM dendrimer was previously dried from methanol at 50 C to yield 4 mg. 4 mg of PAM G=5 was dissolved in 50 ul to give clear solution of 80 mg/ml stock. PEI>25K was prev. prepared by dialysis at 25K tubing the fraction remaining was pooled and sterile filtered in Exp. F2305A to give 143 mg/ml PEI. The 143 mg/ml stock was diluted 1/50 in H2O to give 2.86 mg/ml.

Table #10 Shows appropriate volume (ul) of plasmid DNA stock to be combined with H2O, followed by addition of carrier, and 20% saline solution to give carrier:plasmid nano-particles in a 0.6% final saline solution of 0.4 ml final volume as shown.

TABLE 10

| Group | Carrier | Carrier vol. | pGWz | saline | H2O |
|---|---|---|---|---|---|
| 1 | PAM dendrimer_30 | 30 | 80 | 12 | 278 |
| 2 | pPEGProt92305_250 | 250 | 40 | 12 | 98 |
| 3 | PEI >25K (F2305A) | 12 | 80 | 12 | 296 |
| 4 | pPEGProt92305_250 | 124 | 20 | 12 | 244 |

Mice in groups 1-4 were injected with 0.3 ml, 0.4 ml, 0.3 ml, and 0.3 ml respectively, of each carrier to give either 60, 40, 60, or 15 ug plasmid and 1.8, 10, 0.03, and 3.7 mg carrier per mouse, respectively, as shown in Table #11. Final mg/Kg were calculated based on total mouse weight. The mass ratio of carrier to DNA was 30, 250, for PAM and pPEGProt carriers and N/P ratio was 3.3 for PEI carrier.

Table #11 shows injection volume, final [carrier], final [plasmid] for in-vivo study.

TABLE 11

| | | In-Vivo | In-Vivo | In-Vivo | |
|---|---|---|---|---|---|
| | Inject. | carrier | Carrier | Final ug | In-Vivo |
| Carrier | vol. | Tot. mg | mg/Kg | plasmid | result |
| PAM_30 | 0.30 | 1.8 | 69 | 60 | healthy |
| pPEGProt_250 | 0.40 | 10 | 385 | 40 | toxicity |
| PEI >25K | 0.30 | 0.026 | 1.0 | 60 | healthy |
| pPEGProt_250 | 0.30 | 3.72 | 143 | 15 | healthy |

Forty-eight hours post treatment, mice were euthenized and liver, kidney, spleen, lung, heart removed, rinsed with ice cold PBS and stored ice-cold. Containers+organ were then weighed. Lysis buffer was prepared by combining 2 ml of 5× reporter lysis buffer (Promega) with 0.2 ml of protease inhibitor cocktail (Sigma-Aldrich) and 7.8 ml H2O. Ice-cold lysis buffer was added to whole tissue sample and tissues were frozen (−85 C) and thawed twice, briefly vortexed and centrifuged 10 min at 4 C. Ten microliters (10 ul) of supernatant was then combined with 40 ul luciferase assay reagent. Bioluminescent light was detected on the Synergy HT1, BioteK Inc. For control, firefly lantern luciferase w/luciferin 1× stock frozen −85 C, Sigma-Aldrich) was diluted 1/200 in ice cold water or lysis buffer and 20 ul was combined with 60 ul of water or lysis buffer.

### Example 27

Ex-vivo gene therapy model utilizing pPEGProtamine:pGWzluc nano-particles. Green monkey kidney cells (COS7) were transfected with pPEGProt:pGWzluc nano-particles (EX 11905) and were transplanted into the mouse as a validation of ex-vivo gene therapy model. The COS7 cells expressed the firefly luciferase gene in-vivo and bioluminescence from the luciferase protein was detected in mouse lung, liver, and kidney.

Procedure: COS7 cells were previously grown to confluency in 75 cm2 flask in DMEM media w10% FBS under high humidity atmosphere and 5% CO2. Carrier:DNA nano-particles were prepared by combining fifty microliters (50 ul) of pPEGProtamine92305 24 mg/ml, 1.86 ml H2O, 10 ul of 1 mg/ml pGWzluc plasmid (encoding firefly luciferase gene), and 75 ul of PEGHQ 5.34 mM in a total volume of 2 ml. The nano-particles were diluted in 8 ml final of 1×DMEM media and incubated with COS7 cells. After 2.5 days, cells were removed from the flask by scraping and prepared as a mono dispersed suspension by repeated passaging through a 5 ml pipet. The cells were washed twice with buffered saline to remove media, pelleted, and counted with a hemocytometer. Cell density was 950,000 cells/ml. The cell suspension containing 760,000 cells was then administered to a white mouse via tail vein injection, and after 1 hour, the mouse was euthenized, and the liver, spleen, kidney's, lung, and heart removed, washed in buffered saline, and weighed. Tissues were frozen at −85 C for 24 hours, thawed, and homogenized with lysis buffer. Tissue homogenates were centrifuged for 30 min at 14000 rcf, 25 C, and 20 ul of supernatant was assayed with 80 ul of luciferase assay reagent (Promega, Madison Wis.). Bioluminescence from the expressed gene was detected on a Synergy HT1 spectrophotometer, BioteK Inc. Bioluminescence was highest in the kidney, followed by the liver and lung.

What is claimed is:

1) A shielded micelle composition suitable for the delivery of polynucleotides to a location within a cell, comprising the formula PEG[LX]yQ, in which PEG is an amphiphilic, multi-branched PEG of between 2000 to 500000 daltons, [LX] is a pendant arm, y is the number of pendant arms ranging from between 6 and 500 per mole of multi-branched PEG, and Q is a polynucleotide covalently bound to [LX], such that the mole ratio of Q ranges from between 0.01:1 and 1:1 per mole of [LX].

2) The composition of claim 1 in which X is a functional group selected from a hydroxyl, amine, carbonyl, aldehyde, amide, hydrazine, azide, maleimide, thiol, leaving group, or succinimide.

3) The composition of claim 1 in which the polynucleotide (Q) is covalently attached to [LX] via the 3′ or 5′ end.

4) The composition of claim 1 in which LX is grafted to the backbone of the polynucleotide.

5) The composition of claim 1 where a mole ratio of between 0.01:1 and 1:1 of the branching chain (LX) is grafted to a sulfate, carbonate, phosphate, lipid, vitamin, cholesterol, fatty acid or a combination thereof.

6) The composition of claim 1 in which L is an alkyl chain, a PEG, or a combination thereof.

7) The composition of claim 1, further comprising a membrane permeabilizing agent, selected from chloroquine, hydroxychloroquine, primaquine, or trioxsalen and said agent comprises between 1:1 and 200:1 molar ratio to PEG.

8) The composition of claim 1, in which [LX] is conjugated to a cell targeting peptide, a protamine, or a cationic peptide.

9) The composition of claim 1, further comprising a cellular targeting agent, which is conjugated to [LX] and is selected from antibody, antibody fragments, transferrin, or folic acid.

10) The composition of claim 1, further comprising a mixed micelle formulation containing between 0.01:1 and 50:1 weight:weight ratio of linear PEG to multi-branched PEG, in which linear PEG is conjugated to a hydroxychloroquine, chloroquine, primaquine, or a molecule containing between one and ten nitrogens.

11) A shielded micelle composition suitable for the delivery of polynucleotides to a location within a cell, comprising the formula PEG[LX]yN, in which PEG is an amphiphilic, multi-branched PEG backbone of 2000 to 500000 daltons, [LX] is a pendant arm, y is the number of pendant arms ranging from 6 to 500 per mole of multi-branched PEG, and N is a nitrogen rich moiety covalently bonded to [LX].

12) The composition of claim 11 further comprising a polynucleotide, Q, that is non-covalently bonded to N, such that Q comprises between 1:1 and 400:1 per mole of multi-branched PEG.

13) The composition of claim 12 in which N is conjugated to [LX] at a mole ratio of between 0.01:1 to 100:1 of PEG and N is a hydrazine, and said nitrogens are available for associating with Q, and the polynucleotide Q is non-covalently coupled with N.

14) The composition of claim 12 in which X comprises a molar ratio of 0.01:1 to 1:1 of [LX] and X is a lipid.

15) The composition of 11 in which N comprises between 0.01:1 and 200:1 mole ratio to moles of multi-branched PEG and N is a branched polyethylenimine, and said amines are sufficiently available for association with Q, and the polynucleotide Q is non-covalently bonded to N.

16) The composition of 11 in which N comprises between 0.01:1 and 200:1 mole ratio of multi-branched PEG and N is a polyamido amine.

17) The composition of claim 11 in which N is a protamine.

18) The composition of claim 12 in which [LX] is grafted to an antibody.

19) The claim of 12 in which X is selected from a chloroquine.

20) The claim of 12 further comprising a mixed-micelle composition of between 0.01:1 and 50:1 weight:weight ratio of linear PEG to multi-branched PEG, in which linear PEG is conjugated to hydroxychloroquine.

21) The claim of 12 in which the N comprises a DNA intercalator.

22) The claim of 12 further comprising a mixed micelle composition of between 0.01:1 and 10:1 weight:weight ratio of linear PEG to multi-branched PEG, and linear PEG is conjugated to a DNA intercalator.
